SEQUENCE LISTING (2 sequences)

SEQ ID NO: 1 - DNA, 40 bp, Plasmodium falciparum
catgccatgggccagaatgtgattaatactgtgaagggaa

SEQ ID NO: 2 - DNA, 37 bp, Plasmodium falciparum
cgcggatccgttacattgtgaagtgtcttgtgtagtc

(19) United States
(12) Patent Application Publication
(10) Pub. No.: US 2017/0044150 A1
(43) Pub. Date: Feb. 16, 2017

(54) IDENTIFICATION OF COMPOUNDS WHICH INHIBIT ATG8-ATG3 PROTEIN-PROTEIN INTERACTION AND THEIR USE AS ANTIPARASITICAL AGENTS

(71) Applicant: THE JOHNS HOPKINS UNIVERSITY, Baltimore, MD (US)

(72) Inventors: Jürgen Bosch, Pikesville, MD (US); Adelaide Ulrike P. Hain, Baltimore, MD (US); Caren Freel Meyers, Towson, MD (US); David Bartee, Baltimore, MD (US)

(21) Appl. No.: 15/306,500

(22) PCT Filed: Apr. 27, 2015
(86) PCT No.: PCT/US2015/027690

§ 371 Date: Oct. 25, 2016

Related U.S. Application Data
(60) Provisional application No. 61/984,315, filed on Apr. 25, 2014.

(51) Int. Cl. C07D 417/04; A61K 45/06; A61K 31/443; A61K 31/4436; A61K 31/437; A61K 31/4155; A61K 31/519; A61K 31/40; A61K 31/497; A61K 31/506; A61K 31/4439; A61K 31/4525

(52) U.S. Cl. CPC C07D 417/04; A61K 31/4439; A61K 45/06; A61K 31/443; A61K 31/4436; A61K 31/4525; A61K 31/4155; A61K 31/519; A61K 31/40; A61K 31/497; A61K 31/506; A61K 31/437

(57) ABSTRACT

The present invention provides compounds or a pharmaceutically acceptable salts, solvates, stereoisomers, or prodrugs thereof which can block the Atg8-Atg3 protein-protein interaction, which is associated with autophagy in apicomplexan organisms. Pharmaceutical compositions comprising these compounds and their use for the suppression and treatment of various parasitical diseases are also provided.

### REFERENCE TO RELATED APPLICATIONS

This application claims the benefit of U.S. Provisional Patent Application No. 61/984,315, filed on Apr. 25, 2014, which is hereby incorporated by reference for all purposes as if fully set forth herein.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Apr. 27, 2015, is named P12765-02_ST25.txt and is 656 bytes in size.

### BACKGROUND OF THE INVENTION

The malaria parasite, Plasmodium, is a major public health burden in the developing world and despite the existence of antimalarial treatment active in blood stages of infection, there is a continual need for novel drug design as the parasite develops resistance to current treatments. Additionally, treatment for the hypnozoite-causing species, P. vivax, requires primaquine, which has severe side effects and causes hemolysis in patients with glucose-6-phosphate dehydrogenase deficiency.

Atg8 is the ubiquitin-like (Ubl) protein necessary for formation and maturation of autophagosomes in autophagy in Eukarya. In yeast and mammals, Atg8 is lipidated to the autophagosome membrane, but in Plasmodium, Atg8 is partially conjugated to the membrane of the apicoplast under nonstarvation conditions. The apicoplast is a non-photosynthetic chloroplast-like organelle present in Apicomplexans and is essential for isoprenoid synthesis. Under starvation conditions, Atg8 relocates to acidic vesicles with Rab7 in and near the food vacuole. Atg8 is essential to the Plasmodium parasite and has been proposed as a target for antimalarial drug design.

In most eukaryotes lipidation of Atg8 to phosphatidylethanolamine (PE) in membranes normally requires proteolytic processing of the C-terminus of Atg8 by Atg4 and activation via ATP followed by intermediate thioester bond formation with the E1-activating enzyme Atg7. Atg8 is then transferred to its E2-like conjugating enzyme Atg3, forming a second thioester intermediate before being conjugated to the nitrogen of PE (FIG. 1). This process also requires noncovalent interaction between Atg8 and Atg3 through a well-characterized Atg8-interacting motif (AIM) in Atg3 and through the W/L-site of Atg8. Notably, in Plasmodium, Atg8 is synthesized with a C-terminal glycine and therefore does not require activation by Atg4.

Thus, discovery of novel compounds in antimalarial drug development is essential for future intervention strategies for malaria and other parasitical disease.

### SUMMARY OF THE INVENTION

The present inventors previously elucidated the protein crystal structure of P. falciparum Atg8 bound to a peptide corresponding to PfAtg3's AIM (PDB code 4EOY) (J. Struct. Biol. 2012, 180, 551-562). Regions of diversity exist between the human and Plasmodium system that the inventors now show to be exploitable through small molecule inhibition. Mutational and interaction studies provided herein show that the Plasmodium Atg8-Atg3 interaction requires Atg8's W/L site as well as the apicomplexan loop on Atg8 (residues 67-76), termed the A-loop.

As such, the present invention provides the identification of a class of compounds that inhibit the Apicomplexan (Plasmodium) Atg8-Atg3 interaction as well as inhibiting in vitro growth of P. falciparum in blood- and liver-stage assays, presumably through prevention of PfAtg8 lipidation.

In accordance with an embodiment, the present invention provides a compound of formula I:

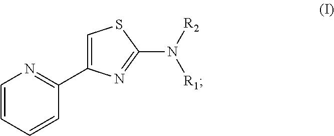

wherein R₁ is H or a C₁-C₃ alkyl, and R₂ is a substituent having the formula of formula II:

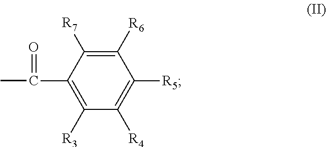

wherein R₃, R₄, R₆, and R₇ each independently represent H or C₁-C₃ alkyl, and R₅ independently represents hydrogen, C₁-C₆ alkyl, C₁-C₆ alkylamino C₁-C₆ alkyl, C₁-C₆ dialkylamino C₁-C₆ alkyl, C₁-C₆ alkylthio C₁-C₆ alkyl, C₁-C₆ alkylsulfonyl C₁-C₆ alkyl, hydroxy C₁-C₆ alkyl, C₁-C₆ alkoxy, C₁-C₆ dialkoxy, C₁-C₆ alkoxy C₁-C₆ alkyl, C₃-C₈ cycloalkyl, heterocyclyl, C₁-C₆ alkylamino, di C₁-C₆ alkylamino, C₁-C₆ alkylthio, C₂-C₆ alkenylthio, C₂-C₆ alkynylthio, C₂-C₆ acyloxy, thio C₂-C₆ acyl, amido, and sulphonamido, and C₁-C₆ alkyl, and C₂-C₆ alkenyl, C₂-C₆ alkynyl; wherein each alkyl moiety may be unsubstituted or substituted with one or more substituents selected from the group consisting of halo, hydroxy, carboxy, phosphoryl, phosphonyl, phosphono C₁-C₆ alkyl, carboxy C₁-C₆ alkyl, dicarboxy C₁-C₆ alkyl, C₁-C₆ dialkyl, dicarboxy halo C₁-C₆ alkyl, sulfonyl, cyano, nitro, alkoxy, alkylthio, acyl, acyloxy, thioacyl, acylthio, aryloxy, amino, alkylamino, dialkylamino, trialkylamino, arylalkylamino, guanidino, aldehydo, ureido, and aminocarbonyl; or a pharmaceutically acceptable salt, solvate, stereoisomer, or a prodrug thereof.

In accordance with another embodiment, the present invention provides a use of a compound of formula I:

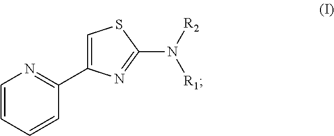

wherein R₁ is H or a C₁-C₃ alkyl, and R₂ is a substituent having the formula of formula II:

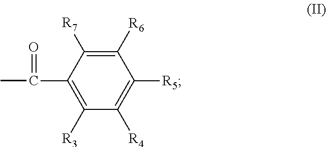

wherein R₃, R₄, R₆, and R₇ each independently represent H or C₁-C₃ alkyl, and R₅ independently represents hydrogen, C₁-C₆ alkyl, C₁-C₆ alkylamino C₁-C₆ alkyl, C₁-C₆ dialkylamino C₁-C₆ alkyl, C₁-C₆ alkylthio C₁-C₆ alkyl, C₁-C₆ alkylsulfonyl C₁-C₆ alkyl, hydroxy C₁-C₆ alkyl, C₁-C₆ alkoxy, C₁-C₆ dialkoxy, C₁-C₆ alkoxy C₁-C₆ alkyl, C₃-C₈ cycloalkyl, heterocyclyl, C₁-C₆ alkylamino, di C₁-C₆ alkylamino, C₁-C₆ alkylthio, C₂-C₆ alkenylthio, C₂-C₆ alkynylthio, C₂-C₆ acyloxy, thio C₂-C₆ acyl, amido, and sulphonamido, and C₁-C₆ alkyl, and C₂-C₆ alkenyl, C₂-C₆ alkynyl; wherein each alkyl moiety may be unsubstituted or substituted with one or more substituents selected from the group consisting of halo, hydroxy, carboxy, phosphoryl, phosphonyl, phosphono C₁-C₆ alkyl, carboxy C₁-C₆ alkyl, dicarboxy C₁-C₆ alkyl, C₁-C₆ dialkyl, dicarboxy halo C₁-C₆ alkyl, sulfonyl, cyano, nitro, alkoxy, alkylthio, acyl, acyloxy, thioacyl, acylthio, aryloxy, amino, alkylamino, dialkylamino, trialkylamino, arylalkylamino, guanidino, aldehydo, ureido, and aminocarbonyl; or a pharmaceutically acceptable salt, solvate, stereoisomer, or a prodrug thereof, as an inhibitor of lipidation of the Atg8 protein in an apicomplexan organism.

In accordance with a further embodiment, the present invention provides a use of the pharmaceutical composition comprising the compound of formula I:

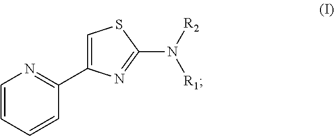

wherein R₁ is H or a C₁-C₃ alkyl, and R₂ is a substituent having the formula of formula II:

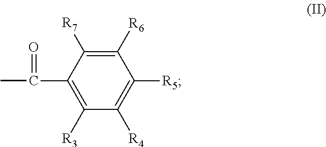

wherein R₃, R₄, R₆, and R₇ each independently represent H or C₁-C₃ alkyl, and R₅ independently represents hydrogen, C₁-C₆ alkyl, C₁-C₆ alkylamino C₁-C₆ alkyl, C₁-C₆ dialkylamino C₁-C₆ alkyl, C₁-C₆ alkylthio C₁-C₆ alkyl, C₁-C₆ alkylsulfonyl C₁-C₆ alkyl, hydroxy C₁-C₆ alkyl, C₁-C₆ alkoxy, C₁-C₆ dialkoxy, C₁-C₆ alkoxy C₁-C₆ alkyl, C₃-C₈ cycloalkyl, heterocyclyl, C₁-C₆ alkylamino, di C₁-C₆ alkylamino, C₁-C₆ alkylthio, C₂-C₆ alkenylthio, C₂-C₆ alkynylthio, C₂-C₆ acyloxy, thio C₂-C₆ acyl, amido, and sulphonamido, and C₁-C₆ alkyl, and C₂-C₆ alkenyl, C₂-C₆ alkynyl; wherein each alkyl moiety may be unsubstituted or substituted with one or more substituents selected from the group consisting of halo, hydroxy, carboxy, phosphoryl, phosphonyl, phosphono C₁-C₆ alkyl, carboxy C₁-C₆ alkyl, dicarboxy C₁-C₆ alkyl, C₁-C₆ dialkyl, dicarboxy halo C₁-C₆ alkyl, sulfonyl, cyano, nitro, alkoxy, alkylthio, acyl, acyloxy, thioacyl, acylthio, aryloxy, amino, alkylamino, dialkylamino, trialkylamino, arylalkylamino, guanidino, aldehydo, ureido, and aminocarbonyl; or a pharmaceutically acceptable salt, solvate, stereoisomer, or a prodrug thereof, in an effective amount, as a medicament, preferably as a medicament for the treatment of a apicomplexan infection in a subject.

In accordance with an embodiment, the present invention provides a pharmaceutical composition comprising one or more of the following compounds selected from the group consisting of:

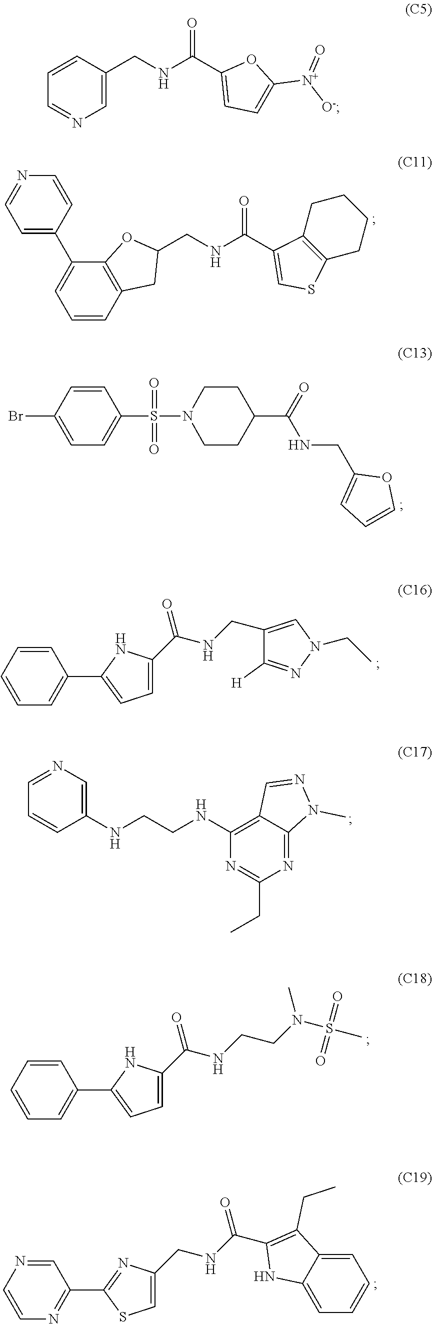

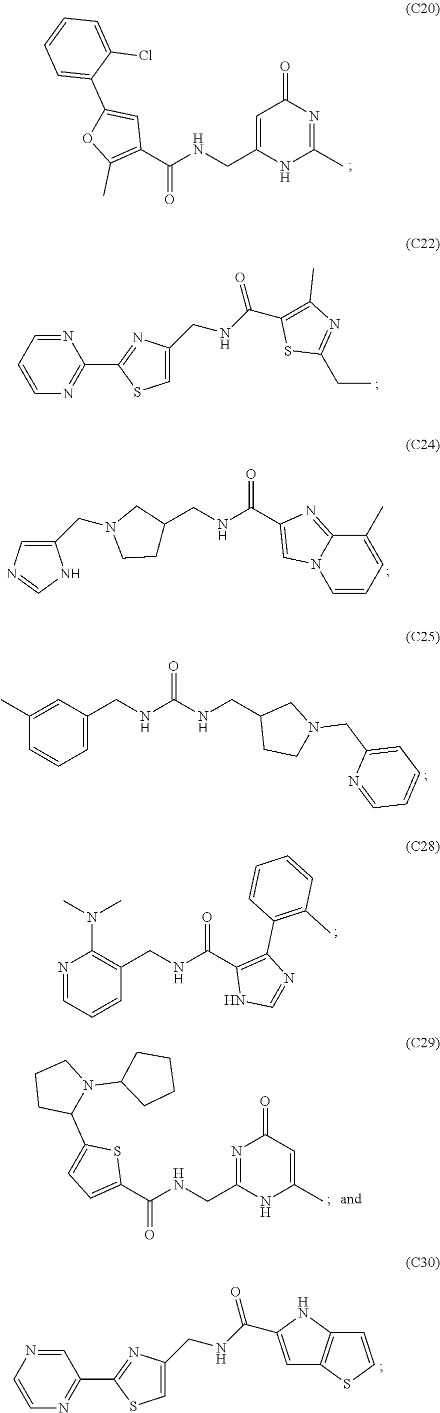

or a pharmaceutically acceptable salt, solvate, stereoisomer, or a prodrug thereof, and a pharmaceutically acceptable carrier.

In accordance with another embodiment, the present invention provides the use of a pharmaceutical composition comprising one or more of the following compounds selected from the group consisting of:

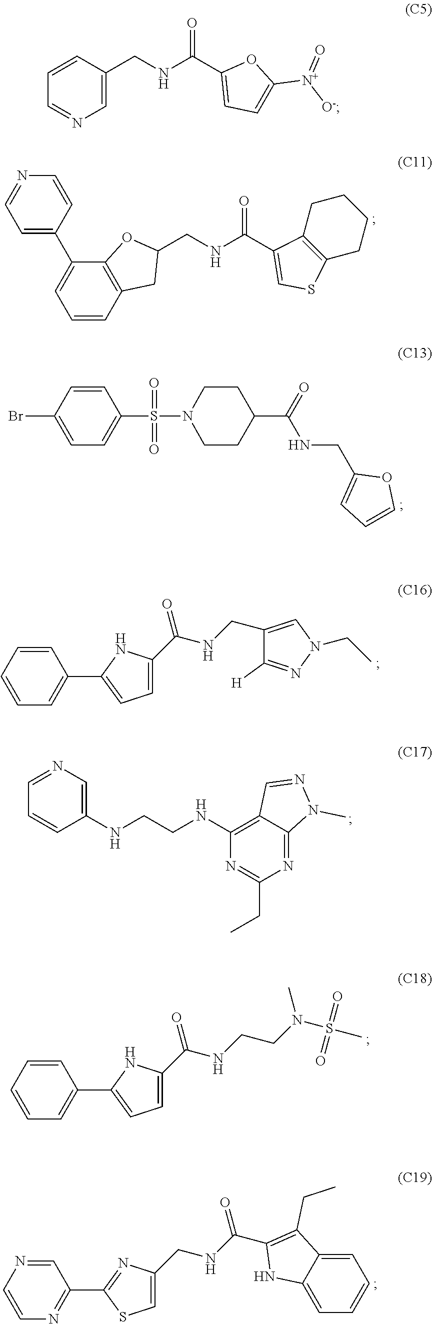

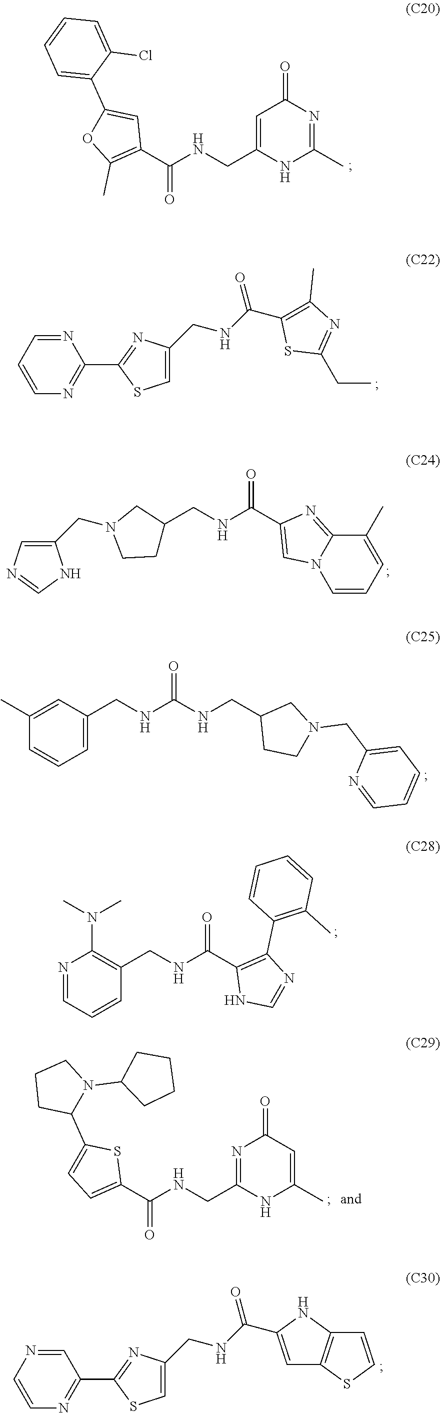

or a pharmaceutically acceptable salt, solvate, stereoisomer, or a prodrug thereof, as an inhibitor of lipidation of the Atg8 protein in an apicomplexan organism.

In accordance with a further embodiment, the present invention provides the use of a pharmaceutical composition comprising one or more of the following compounds selected from the group consisting of:

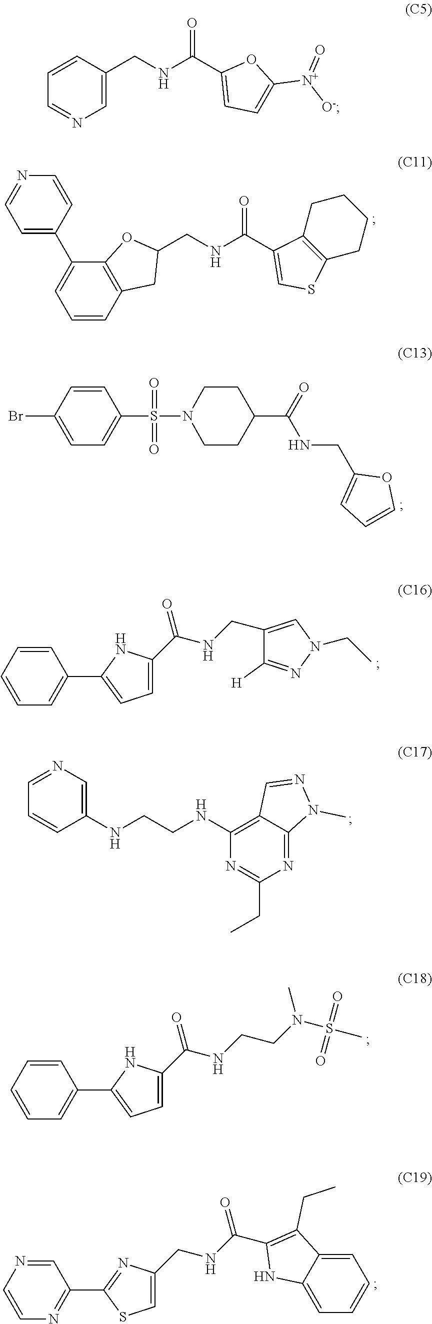

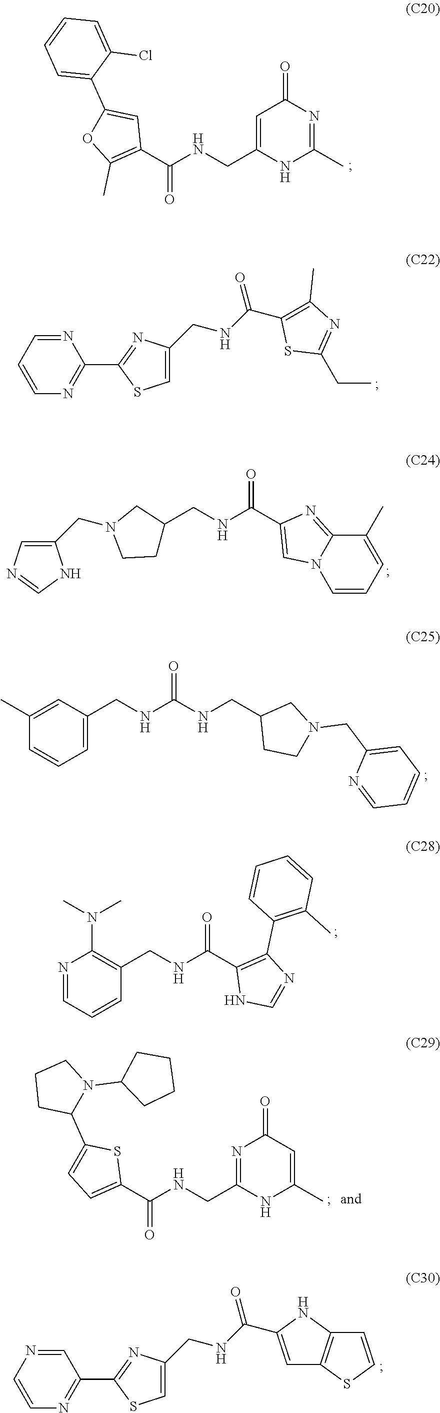

or a pharmaceutically acceptable salt, solvate, stereoisomer, or a prodrug thereof, in an effective amount, as a medicament, preferably as a medicament for the treatment of an apicomplexan infection in a subject.

In accordance with another embodiment, the present invention provides a use of the pharmaceutical composition comprising the compounds described herein, and at least one additional biologically active agent for the treatment of an apicomplexan infection in a subject.

### BRIEF DESCRIPTION OF THE DRAWINGS

FIG. 1 illustrates an overview of the Atg8 conjugation pathway in autophagy. 1A. Visual representation of the localization of Atg8 in autophagy. Atg8, represented by the rectangle, is essential to the elongation of the autophagosomal membrane. 1B. Generic conjugation pathway shown for yeast system. In Plasmodium, Atg8 is synthesized with a C-terminal glycine that does not require proteolytic processing for activation. Red line indicates step of pathway targeted by some of the inhibitors of the present invention.

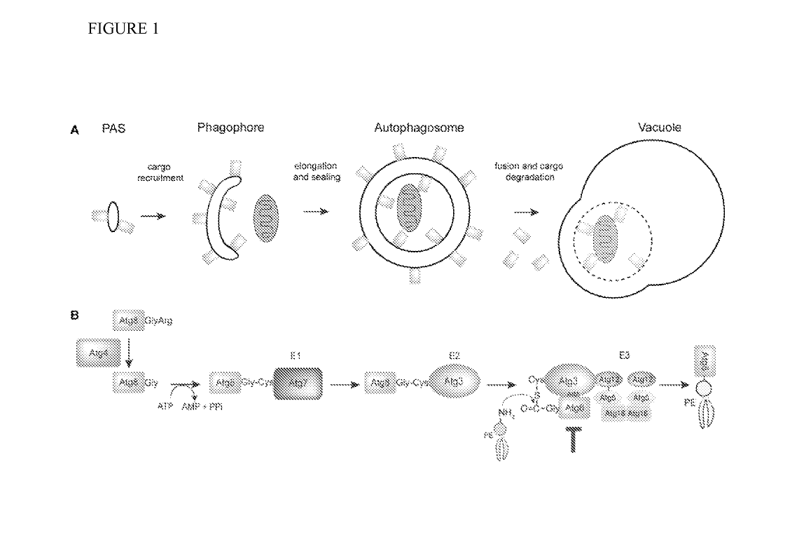

FIG. 2 depicts the identification of a common scaffold that inhibits Atg8-Atg3 from the MMV malaria box screen. 2A. Primary screen of MMV Malaria Box with SPR competition assay. Gray box denotes compounds meeting the threshold of greater than 25% inhibition. 2B. Bar graph showing inhibition of hits in primary screen, denoted by compound number. 2C. Dose-dependent inhibition of PfAtg8-PfAtg3 interaction by MMV compounds. Inhibition was measured with increasing amount of compound in SPR competition screen. Mean and SD of 3 injections are shown. 2D. Thermal stability assay of PfAtg8CM with the three MMV hits. Error bars show SD of 3 measurements.

FIG. 3 shows the identification of the compound 1 binding site on PfAtg8CM. 3A. Binding of compound 1 to PfAtg8. His12-PfAtg8CM was immobilized onto a nickel-charged NTA SPR chip. Compound 1 was injected over variants in two separate runs at 4 concentrations. Graph shows mean±SD of binding from compound 1. 3B. In silico docking of PTA compounds to PfAtg8CM (PDB code 4EOY). Overall structure of PfAtg8 with W- and L-site and A-loop demarcated. Inset shows best poses for each compound. The predicted pose for unconstrained docking is shown in cyan. Docking was also performed constraining to the carbonyl of Lys47, located between the W- and L-site. Predicted pose for constrained docking is shown in green. For compound 1, an alternate pose, highly ranked in the unconstrained docking and enriched in top ten poses is shown in magenta.

FIG. 4 depicts P. falciparum and P. yoelii Atg8 structural differences. PfAtg8 (PDB code 4EOY) is shown with surface and cartoon representation. Amino acid changes between the species are shown in red with P. falciparum letter and numbering followed by P. yoelii. W-site, L-site, and A-loop pockets are shown in mesh in cyan, purple, and green, respectively. P. falciparum Atg8 pocket sizes were calculated with OpenEye VIDA visualization software (eyesopen.com).

FIG. 5 illustrates the effect of compound 1 treatment on the development of P. falciparum 3D7 GFP parasite in HC-04 cells in vitro. 5A. Flow cytometry based detection of Annexin-V positive and PI-positive cells in hepatocyte cultures treated with compound 1 (as described in the Examples). Dot plot graphs demonstrate representative pattern of staining, bar graphs show summary (mean±SD) of viable cell detection obtained in three independent hepatocyte cultures. 5B. Viable infected hepatocytes (GFP+/PI−) were detected by flow cytometry in HC-04 cultures 72 hours post infection with P. falciparum. Dot plot graphs demonstrate representative pattern of staining, numbers reflect percentages of GFP positive cells in total PI negative cell populations. 5C. Summary (mean±SD) of viable infected cell detection obtained in three independent hepatocyte cultures. D. GFP-specific fluorescence was assessed in infected cultures exposed to compound 1 or DMSO for 72 hours. Histograms demonstrate one representative staining pattern, numbers reflect mean fluorescence intensity (MFI) in GFP-positive populations. Bar graphs reflect GFP-specific MFI (mean±SD) in viable cell population detected in three independent hepatocyte cultures.

FIG. 6 depicts the effect of compound 1 on PfAtg8 protein levels. 6A. Immunoblot analysis of P. falciparum FCR3 cells treated with DMSO, DMSO and culture media lacking human serum, or 50 μM compound 1 for five hours. Upper blot was probed with antibody against TgAtg8, demonstrated to be cross reactive against PfAtg8. Asterisk indicates migration of PfAtg8 in compound 1-treated cells versus double asterisk indicating migration of PfAtg8 in control and starved cells. Atg8-PE has a faster migration than unlipidated Atg8 with SDS-PAGE. Band intensity of PfAtg8 was normalized to total protein on stained membrane with ImageJ. PfAtg8 was 23 times greater in the compound 1-treated sample than the control. 6B. Dose-dependent immunoblot analysis of P. falciparum treated with DMSO or 5 μM, 25 μM, 50 μM compound 1 for 12 hours. A dotted line represents the migration of lipidated PfAtg8. 6C. Blood smears of P. falciparum after treatment with DMSO or 50 μM compound 1 for five hours, observed at 100× magnification. Representative images for different stages are shown, progressing from ring stage on the left to late schizont on the right.

FIG. 7 shows the analysis of PTA-derivatives. 7A. In silico docking of PTA-derivatives on PfAtg8. Compound 7 (yellow) and compound 9 (green) were docked onto the constrained receptor of PfAtg8^{CM} (PDB code 4EOY) with OpenEye docking suite. 7B. Direct binding of the PTA scaffold to PfAtg8. PfAtg8^{CM} and hLC3 were injected over immobilized compound 8 and binding was measured with SPR. 7C. Superposition of small molecule hits derived from MMV Malaria Box with compound 9. The individual molecular surfaces with their corresponding electrostatic potential are depicted in side and top view. All four molecules share the PTA moiety and were superimposed using ROCS via shape complementarity and Tanimoto color scoring function. The figure was prepared with Vida and rendered in PovRay (povray.org).

FIG. 8 illustrates the validation of compound 7 as starting point for optimization. 8A. Inhibition of PfAtg8-PfAtg3 interaction by compound 9. SPR response of PfAtg8 injected over immobilized PfAtg3 was measured in the presence of increasing concentration of compound 9. IC₅₀ was determined as 2.86 μM. Mean±SD of 3 injections are shown. 8B. Inhibition of blood stage parasites compound 9. SYBR green I assays were used to measure inhibition by CQ, compound 1, and compound 9. Growth inhibition curves are shown for one experiment with IC₅₀ values from two to three experiments in table inset.

FIG. 9 depicts Atg8, a pan-apicomplexan drug target. (A) Atg8 sequence alignment for ten species of Plasmodium (ESPript, global score set to 0.9). The sequence is color-coded to indicate residues involved in ligand interaction sites: magenta, green, and orange correspond to the W-site, L-site, and A-site, respectively. (B) Sequence alignment of the A-loop in several apicomplexan species and human homologues (ESPript, global score set to 0.4). The A-loop is notably absent from the human homologues. (C) Structure of PfAtg8 (PDB: 4EOY) color-coded as in (A), showing the ligand interaction sites. (D) Comparison of PfAtg8 (PDB: 4EOY) and hLC3 (PDB: 2ZJD) structures, illustrating A-loop divergence. Yellow loop corresponds to yellow box delineated in (B). (E) A-loop pocket of PfAtg8 color-coded based on conservation with five apicomplexan homologues. N. caninum and T. gondii Atg8 amino acid sequences are identical.

FIG. 10 shows the generation of an in-silico PfAtg8 receptor for docking studies. (A) PfAtg8 model used in OpenEye docking, with docking restraints indicated by green and magenta spheres. (B) A-loop pocket size characteristics. (C) ChemBridge Core Library properties showing the c Log P distribution, the molecular weight distribution, and the percentage of chiral molecules in the library. (D) Omega2 conformer plot highlighting a significant amount of flexible molecules present in the library. (E) VLS flow diagram and attrition rate at each step.

FIG. 11 depicts protein-protein interaction inhibition assayed via SPR. All binding responses have been normalized to the baseline control at 100%. (A) Schematic of SPR interaction study. The two figures illustrate SPR interactions in the absence (top) and presence (bottom) of compound. (B) Effect of compounds on PfAtg3-PfAtg8 interaction. Green dotted line corresponds to control response. Blue indicates some precipitation of protein or compound observed at 500 μM concentration. (C) Effect of compounds on hAtg3-hLC3 interaction. Green—control; blue—observed precipitation. (D) The TSIS plot for human and P. falciparum data. Human (x-axis) versus Plasmodium (y-axis) normalized SPR binding. Dotted lines are the control response. Gray rectangle encloses compounds within one standard deviation of average inhibited response. Green rectangle encloses favorable area for potential antimalarial molecules.

FIG. 12 depicts compound C25 characterization. (A) IC₅₀ of C25 in blood stage parasites (B) PfAtg8 melting temperature in the presence of increasing concentrations of C25.

FIG. 13 illustrates the C25 FRED docking report. (A) C25 docking pose showing four hydrogen bond interactions with PfAtg8. (B) Schematic representation of the ligand environment and pocket characteristics, showing that most of the C25 ligand occupies the A-loop pocket. A region near the pyrrolidine ring would allow further extension of the ligand. (C) C25 had the second highest docking score of all tested compounds in the filtered ChemBridge core library. (D) The residue fingerprint highlights residues in proximity to the ligand, bolded residues are in contact with the ligand, which are further distinguished by their respective property. (E) Additional properties for ligand optimization guidance are given based on shape, hydrogen bonding interaction, protein and ligand desolvation energies.

FIG. 14 shows the effect of C25 on blood stage parasite development. (A) IC50 of C25 and CQ in blood stage parasites. (B) Western blot indicating shift in PfAtg8 mobility upon addition of C25 corresponding to a shift from a predominantly lipidated (black dotted line) to unlipidated (green dotted line) population. PTA western blot published in (10). (C) Quantification of PfAtg8 on western blot.

FIG. 15 depicts the effect of C25 on liver stage parasite development. (A) Flow cytometry-based detection of P. falciparum 3D7HT-GFP EEFs propagated in the presence of C25 or DMSO as a vehicle control until 96 hrs post-infection. Representative dot plots for each condition are shown. Numbers reflect percentages of viable infected cells (GFP+/PI−). The bar graph gives the mean (+/−SD) of EEF percentages from three independent hepatocyte cultures. Noninfected and DMSO controls published in (10). (B) Representative dot plots illustrating percentages of viable cells (Annexin V−/PI−) detected by flow cytometry in HC-04 cultures treated with C25 or DMSO (vehicle control) for 96 hrs. The bar graph gives the mean (+/−SD) for three independent hepatocyte cultures.

### DETAILED DESCRIPTION OF THE INVENTION

In accordance with one or more embodiments, the present inventors describe the identification of inhibitors from the Malaria Medicine Venture (MMV) Malaria Box against the protein-protein interaction of PfAtg8 with its E2-conjugating enzyme, PfAtg3 by surface plasmon resonance (SPR). Inhibition of the protein-protein interaction of PfAtg8 with PfAtg3 prevents PfAtg8 lipidation with phosphatidylethanolamine (PE). The results of these inventive methods were the discovery of a class of small molecule inhibitors of the PfAtg8-PfAtg3 interaction. These novel small molecule inhibitors of the present invention share a common scaffold and have activity against both blood and liver stages of infection by P. falciparum. The present inventors have derivatized this scaffold into a functional platform, while retaining in vitro parasite growth inhibitory activity for further optimization.

In accordance with an embodiment, the present invention provides a compound of formula I:

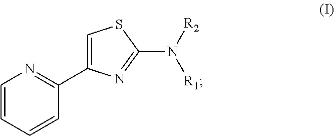

wherein R₁ is H or a C₁-C₃ alkyl, and R₂ is a substituent having the formula of formula II:

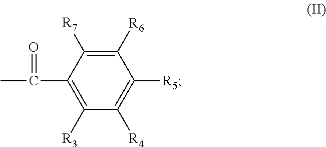

wherein R₃, R₄, R₆, and R₇ each independently represent H or Cₗ-C₃ alkyl, and R₅ independently represents hydrogen, C₁-C₆ alkyl, C₁-C₆ alkylamino C₁-C₆ alkyl, C₁-C₆ dialkylamino C₁-C₆ alkyl, C₁-C₆ alkylthio C₁-C₆ alkyl, C₁-C₆ alkylsulfonyl C₁-C₆ alkyl, hydroxy C₁-C₆ alkyl, C₁-C₆ alkoxy, C₁-C₆ dialkoxy, C₁-C₆ alkoxy C₁-C₆ alkyl, C₃-C₈ cycloalkyl, heterocyclyl, C₁-C₆ alkylamino, di C₁-C₆ alkylamino, C₁-C₆ alkylthio, C₂-C₆ alkenylthio, C₂-C₆ alkynylthio, C₂-C₆ acyloxy, thio C₂-C₆ acyl, amido, and sulphonamido, and C₁-C₆ alkyl, and C₂-C₆ alkenyl, C₂-C₆ alkynyl; wherein each alkyl moiety may be unsubstituted or substituted with one or more substituents selected from the group consisting of halo, hydroxy, carboxy, phosphoryl, phosphonyl, phosphono C₁-C₆ alkyl, carboxy C₁-C₆ alkyl, dicarboxy C₁-C₆ alkyl, C₁-C₆ dialkyl, dicarboxy halo C₁-C₆ alkyl, sulfonyl, cyano, nitro, alkoxy, alkylthio, acyl, acyloxy, thioacyl, acylthio, aryloxy, amino, alkylamino, dialkylamino, trialkylamino, arylalkylamino, guanidino, aldehydo, ureido, and aminocarbonyl; or a pharmaceutically acceptable salt, solvate, stereoisomer, or a prodrug thereof.

In the compounds disclosed herein, including, e.g., the compound of formula I, the “hydrocarbon group” of the “hydrocarbon group which may be substituted” represented by R may be exemplified by a straight-chained or cyclic hydrocarbon group (e.g., an alkyl group, an alkenyl group, an alkynyl group, a cycloalkyl group, an aryl group, an arylalkyl group, alkylol group, hydroxyalkyl group, alkoxy group, alkoxyalkyl group, a branched or straight-chain alkylamino, dialkylamino, or alkyl or dialkylaminoalkyl, or thioalkyl, thioalkenyl, thioalkynyl, aryloxy, thioaryl, thioheteroaryl, acyloxy, thioacyl, amido, sulphonamido, etc.), or the like. Among these, straight-chained or cyclic hydrocarbon groups having 1 to 6 carbon atoms are preferred.

Examples of the “arylalkyl group” preferably include a C₆₋₁₄ arylalkyl group (e.g., a benzyl group, a phenylethyl group, a diphenylmethyl group, a 1-naphthylmethyl group, a 2-naphthylmethyl group, a 2,2-diphenylethyl group, a 3-phenylpropyl group, a 4-phenylbutyl group, a 5-phenylpentyl group, etc.) and the like.

The term “hydroxyalkyl” embraces linear or branched alkyl groups having one to about ten carbon atoms any one of which may be substituted with one or more hydroxyl groups.

The term “alkylamino” includes monoalkylamino. The term “monoalkylamino” means an amino, which is substituted with an alkyl as defined herein. Examples of monoalkylamino substituents include, but are not limited to, methylamino, ethylamino, isopropylamino, t-butylamino, and the like. The term “dialkylamino” means an amino, which is substituted with two alkyls as defined herein, which alkyls can be the same or different. Examples of dialkylamino substituents include dimethylamino, diethylamino, ethylisopropylamino, diisopropylamino, dibutylamino, and the like.

The terms “alkylthio,” “alkenylthio” and “alkynylthio” group mean a group consisting of a sulphur atom bonded to an alkyl-, alkenyl- or alkynyl-group, which is bonded via the sulphur atom to the entity to which the group is bonded.

In accordance with an embodiment, the present invention provides a compound of formula I, as described above, wherein R₁, R₃, R₄, R₆, and R₇ are H.

In accordance with an embodiment, the present invention provides a compound of formula I, as described above, R₁, R₃, R₄, R₆, and R₇ are H and wherein R₅ is a C₁ carboxy group.

In accordance with an embodiment, the present invention provides a compound of formula I, as described above, R₁, R₃, R₄, R₆, and R₇ are H and wherein R₅ is a C₁ alkyl group substituted with a dimethoxy group.

In accordance with an embodiment, the present invention provides a compound of formula I, selected from the group consisting of:

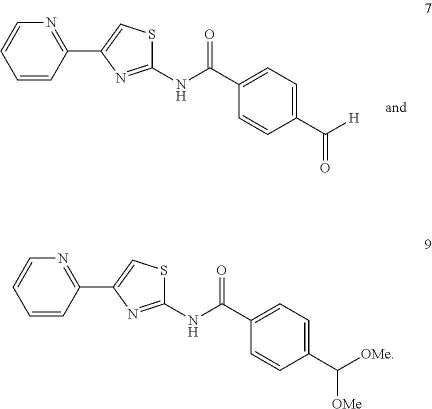 [SMILES: O=Cc1ccc(C(=O)Nc2nc(-c3ccccn3)cs2)cc1 ; COC(OC)c1ccc(C(=O)Nc2nc(-c3ccccn3)cs2)cc1]

In accordance with an embodiment, the present invention provides a pharmaceutical composition comprising one or more of the following compounds selected from the group consisting of:

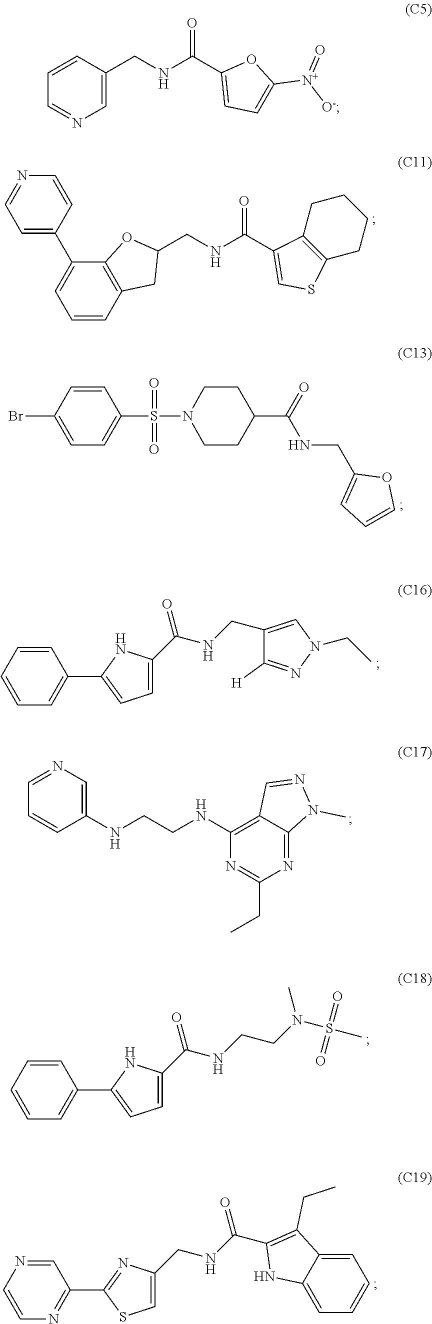

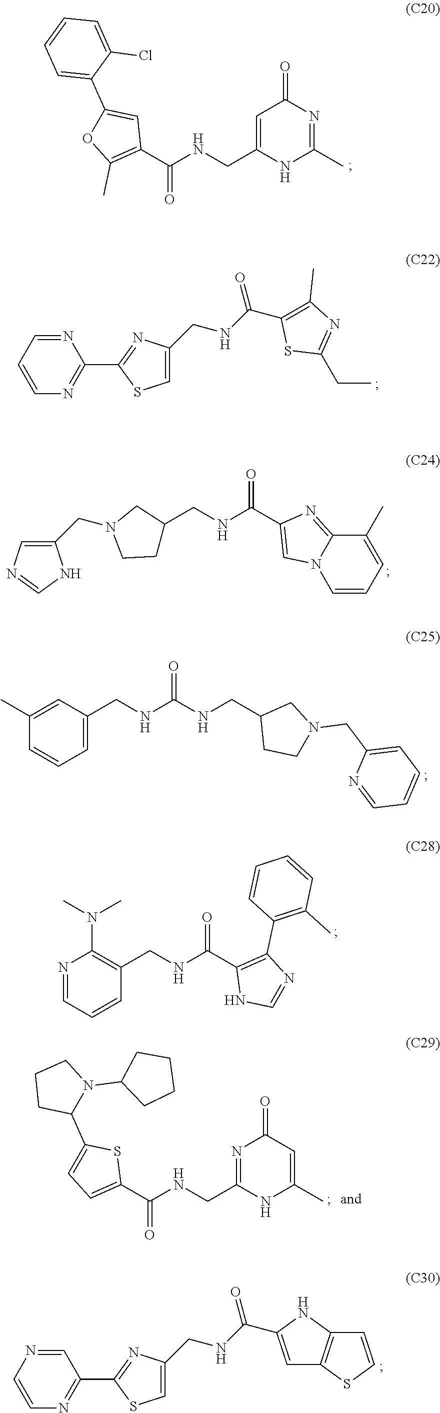

or a pharmaceutically acceptable salt, solvate, stereoisomer, or a prodrug thereof, and a pharmaceutically acceptable carrier.

In accordance with another embodiment, the present invention provides the use of a pharmaceutical composition comprising one or more of the following compounds selected from the group consisting of:

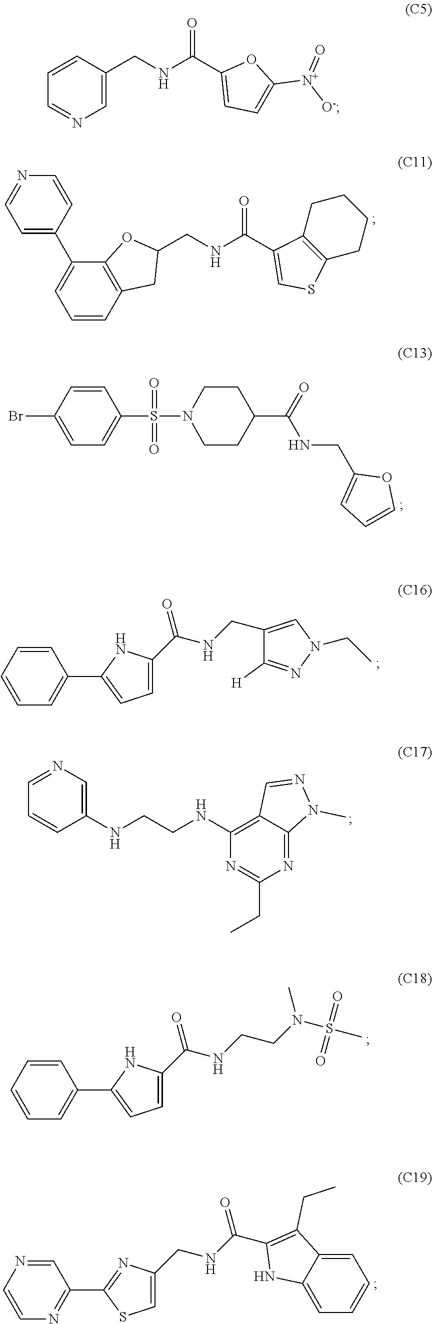

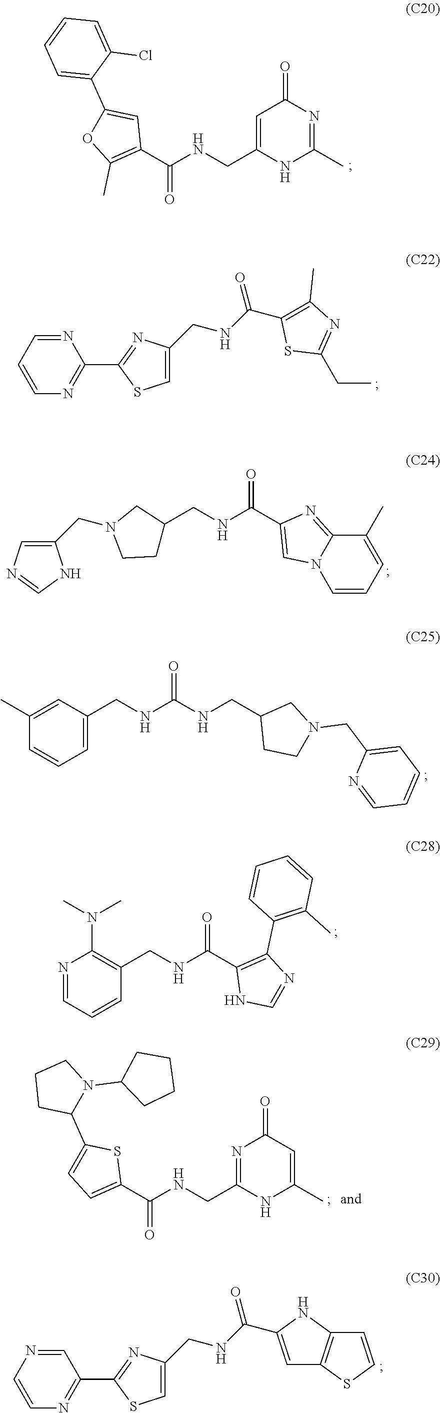

or a pharmaceutically acceptable salt, solvate, stereoisomer, or a prodrug thereof, as an inhibitor of lipidation of the Atg8 protein in an apicomplexan organism.

In accordance with a further embodiment, the present invention provides the use of a pharmaceutical composition comprising one or more of the following compounds selected from the group consisting of:

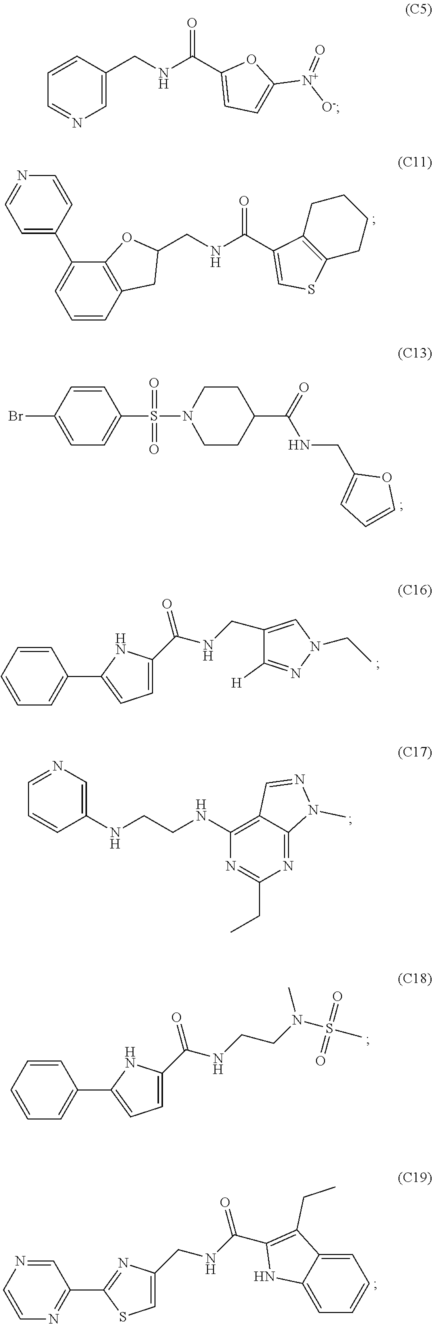

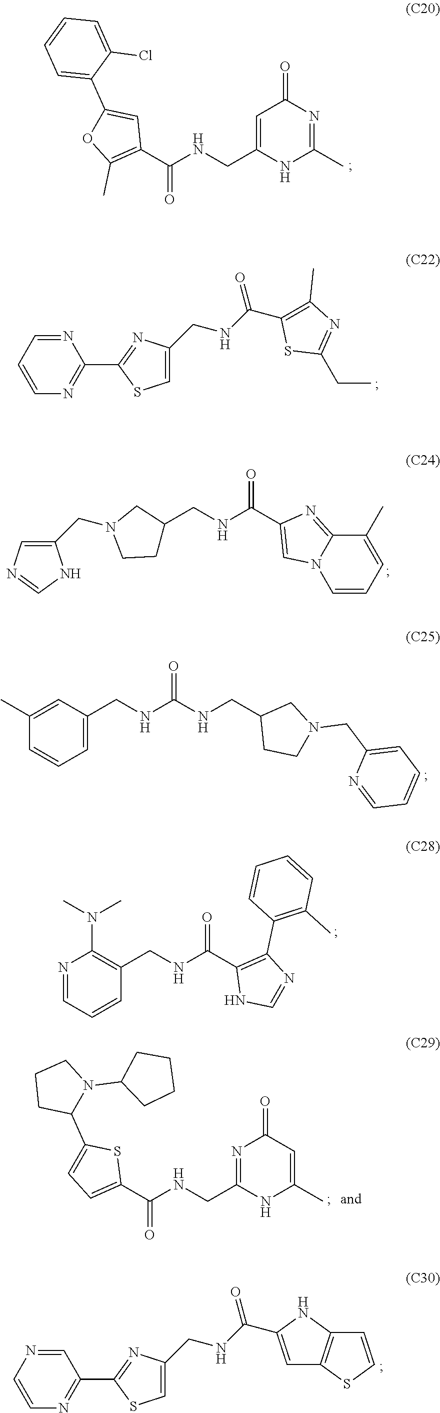

or a pharmaceutically acceptable salt, solvate, stereoisomer, or a prodrug thereof, in an effective amount, as a medicament, preferably as a medicament for the treatment of an apicomplexan infection in a subject.

In accordance with another embodiment, the present invention provides a use of the pharmaceutical composition comprising the compounds described herein, and at least one additional biologically active agent for the treatment of an apicomplexan infection in a subject.

In accordance with an embodiment, the present invention provides a pharmaceutical composition comprising the compounds described herein, or a salt, solvate, stereoisomer, or prodrug thereof, and a pharmaceutically acceptable carrier.

Included within the compounds of the present invention are the tautomeric forms of the disclosed compounds, isomeric forms including diastereoisomers, and the pharmaceutically-acceptable salts thereof. The term “pharmaceutically acceptable salts” embraces salts commonly used to form alkali metal salts and to form addition salts of free acids or free bases. Examples of acids which may be employed to form pharmaceutically acceptable acid addition salts include such inorganic acids as hydrochloric acid, sulphuric acid and phosphoric acid, and such organic acids as maleic acid, succinic acid and citric acid. Other pharmaceutically acceptable salts include salts with alkali metals or alkaline earth metals, such as sodium, potassium, calcium and magnesium, or with organic bases, such as dicyclohexylamine. Suitable pharmaceutically acceptable salts of the compounds of the present invention include, for example, acid addition salts which may, for example, be formed by mixing a solution of the compound according to the invention with a solution of a pharmaceutically acceptable acid, such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. All of these salts may be prepared by conventional means by reacting, for example, the appropriate acid or base with the corresponding compounds of the present invention.

Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicines, the salts of the compounds of the present invention should be pharmaceutically acceptable salts. Other salts may, however, be useful in the preparation of the compounds according to the invention or of their pharmaceutically acceptable salts.

In addition, embodiments of the invention include hydrates of the compounds of the present invention. The term “hydrate” includes but is not limited to hemihydrate, monohydrate, dihydrate, trihydrate and the like. Hydrates of the compounds of the present invention may be prepared by contacting the compounds with water under suitable conditions to produce the hydrate of choice.

As defined herein, in one or more embodiments, “contacting” means that the one or more compounds of the present invention are introduced into a sample having at least one apicomplexan organism, e.g. an Plasmodium organism, including for example, Plasmodium falciparum, and appropriate enzymes or reagents, in a test tube, flask, tissue culture, chip, array, plate, microplate, capillary, or the like, and incubated at a temperature and time sufficient to permit binding of the at least one compounds of the present invention to interact with the organism.

Thus, in accordance with an embodiment, the present invention provides the compound of formula I, or a pharmaceutical composition comprising the compound of formula I, as described herein, as an inhibitor of lipidation of the Atg8 protein in an apicomplexan organism.

As used herein, the term “apicomplexan organism” means an organism that is a member of an extremely large and diverse group (>5000 named species). Seven species infect humans. Plasmodium, as the causative agent of malaria, has the greatest impact on human health. Babesia is a relatively rare zoonotic infection. The other five species are all classified as coccidia. However, recent molecular data indicates that Cryptosporidium is more closely related to the gregarines than to the coccidia. The coccidia are generally considered opportunistic pathogens and are often associated with AIDS, and generally, in immune-compromised patients such as treated cancer patients, post-operative patients, and pregnant women, for example. Several apicomplexan parasites are also important in terms of veterinary medicine and agriculture. Most notable are Babesia and Theileria in cattle and Eimeria in poultry. The apicomplexa have complex life cycles that are characterized by three distinct processes: sporogony, merogony and gametogony.

In a further embodiment, the present invention provides a method of treating a apicomplexan infection in a subject, the method comprising administering to the subject, a pharmaceutical composition comprising at least one compound of the present invention. In another embodiment, the method comprises administering to the subject, a pharmaceutical composition comprising at least one compound of the present invention, and at least one other compound suitable for use in treating an apicomplexan infection, with a pharmaceutically acceptable carrier, in an effective amount to inhibit, suppress or treat symptoms of the apicomplexan infection.

In accordance with an embodiment, the apicomplexan organism treated using the compounds and compositions of the present invention are selected from the group consisting of Plasmodium, Babesia, Cryptosporidium, Cyclospora, Isospora, Eimeria, Theileria and Toxoplasma.

In some embodiments, the apicomplexan infection is a Plasmodium infection and the disease is malaria.

In an embodiment, the pharmaceutical compositions of the present invention comprise the compounds of the present invention together with a pharmaceutically acceptable carrier.

In an embodiment, the at least one other compound suitable for use in treating an apicomplexan infection is an antiparasitical composition. In some embodiments, the antiparasitical compound is an antimalarial compound. Suitable antimalarial compounds for use in treating a Plasmodium infection include, for example, the artemisinins, sulfadoxine, pyrimethamine, doxycycline, azithromycin, atovaquone, tetracycline, other antifolates like trimethoprim, sulfamethoxazole, quinolones, clindamycinand nitazoxanide.

For anti-coccidio drugs, examples include sulfonamides (sulfanilamide, trimethoprim, ethopabate), pyridinoles (clopidol, decoquinate), nitrobenzamides (zoalene), organic arsenicals (roxarsone), nitrofurans (furazolidone, amprolium), quinazolinones (halofuginone), polyether ionophorous antibiotics (monensin, laslocid, salinomycin, narasin), asymmetric (diclazuril) and symmetric (toltrazuril) triazines.

Embodiments of the invention include a process for preparing pharmaceutical products comprising the compounds, salts, solvates or stereoisomers thereof. The term “pharmaceutical product” means a composition suitable for pharmaceutical use (pharmaceutical composition), as defined herein. Pharmaceutical compositions formulated for particular applications comprising the Plasmodium inhibitors of the present invention are also part of this invention, and are to be considered an embodiment thereof.

As used herein, the term “treat,” as well as words stemming therefrom, includes preventative as well as disorder remitative treatment. The terms “reduce”, “suppress” and “inhibit,” as well as words stemming therefrom, have their commonly understood meaning of lessening or decreasing. These words do not necessarily imply 100% or complete treatment, reduction, suppression, or inhibition.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

With respect to pharmaceutical compositions described herein, the pharmaceutically acceptable carrier can be any of those conventionally used, and is limited only by physico-chemical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s), and one which has little or no detrimental side effects or toxicity under the conditions of use. Examples of the pharmaceutically acceptable carriers include soluble carriers such as known buffers which can be physiologically acceptable (e.g., phosphate buffer) as well as solid compositions such as solid-state carriers or latex beads.

The carriers or diluents used herein may be solid carriers or diluents for solid formulations, liquid carriers or diluents for liquid formulations, or mixtures thereof.

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Intravenous vehicles include, for example, fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions.

In addition, in an embodiment, the compounds of the present invention may further comprise, for example, binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmelose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., cremophor, glycerol, polyethylene glycerol, benzlkonium chloride, benzyl benzoate, cyclodextrins, sorbitan esters, stearic acids), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweetners (e.g., aspartame, citric acid), preservatives (e.g., thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates), and/or adjuvants.

The choice of carrier will be determined, in part, by the particular compound, as well as by the particular method used to administer the compound. Accordingly, there are a variety of suitable formulations of the pharmaceutical composition of the invention. The following formulations for parenteral, subcutaneous, intravenous, intramuscular, intraarterial, intrathecal and interperitoneal administration are exemplary, and are in no way limiting. More than one route can be used to administer the compounds of the present invention, and in certain instances, a particular route can provide a more immediate and more effective response than another route.

Suitable soaps for use in parenteral formulations include, for example, fatty alkali metal, ammonium, and triethanolamine salts, and suitable detergents include, for example, (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the compound of the present invention or a salt, solvate or stereoisomer thereof, in solution. Preservatives and buffers may be used. In order to minimize or eliminate irritation at the site of injection, such compositions may contain one or more nonionic surfactants, for example, having a hydrophile-lipophile balance (HLB) of from about 12 to about 17. The quantity of surfactant in such formulations will typically range from about 5% to about 15% by weight. Suitable surfactants include, for example, polyethylene glycol sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Injectable formulations are in accordance with the invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Trissel, 15th ed., pages 622-630 (2009)).

For purposes of the invention, the amount or dose of the compound of the present invention, or a salt, solvate or stereoisomer thereof, administered should be sufficient to effect, e.g., a therapeutic or prophylactic response, in the subject over a reasonable time frame. The dose will be determined by the efficacy of the particular compound and the condition of a human, as well as the body weight of a human to be treated.

The dose of the compound of the present invention, or a salt, solvate or stereoisomer thereof, also will be determined by the existence, nature and extent of any adverse side effects that might accompany the administration of a particular compound. Typically, an attending physician will decide the dosage of the compound with which to treat each individual patient, taking into consideration a variety of factors, such as age, body weight, general health, diet, sex, compound to be administered, route of administration, and the severity of the condition being treated. By way of example, and not intending to limit the invention, the dose of the compound can be about 0.001 to about 100 mg/kg body weight of the subject being treated/day, preferably about 1 mg/kg/day to about 50 mg/kg/day. In some embodiments, the dosage levels of the compounds would be in the range of 10 μM to about 500 μM, preferably about 100 μM to about 300 μM.

Alternatively, the compound of the present invention, or a salt, solvate or stereoisomer thereof, can be modified into a depot form, such that the manner in which the compound is released into the body to which it is administered is controlled with respect to time and location within the body (see, for example, U.S. Pat. No. 4,450,150). Depot forms of compound can be, for example, an implantable composition comprising the compound and a porous or non-porous material, such as a polymer, wherein compound is encapsulated by or diffused throughout the material and/or degradation of the non-porous material. The depot is then implanted into the desired location within the body and the compounds are released from the implant at a predetermined rate.

In one embodiment, the compounds of the present invention, or salts, solvates or stereoisomers thereof, provided herein can be controlled release compositions, i.e., compositions in which the one or more compounds are released over a period of time after administration. Controlled or sustained release compositions include formulation in lipophilic depots (e.g., fatty acids, waxes, oils). In another embodiment the composition is an immediate release composition, i.e., a composition in which all or substantially all of the compound of formula I is released immediately after administration.

In yet another embodiment, the compounds of the present invention can be delivered in a controlled release system. For example, the agent may be administered using intravenous infusion, an implantable osmotic pump, a transdermal patch, or other modes of administration. In an embodiment, a pump may be used. In one embodiment, polymeric materials can be used. In yet another embodiment, a controlled release system can be placed in proximity to the therapeutic target, i.e., the brain, thus requiring only a fraction of the systemic dose (see, e.g., Design of Controlled Release Drug Delivery Systems, Xiaoling Li and Bhaskara R. Jasti eds. (McGraw-Hill, 2006)).

The compounds of the present invention, or salts, solvates or stereoisomers thereof, may also include incorporation of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

In accordance with the present invention, the compounds may be modified by, for example, the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection, than do the corresponding unmodified compounds. Such modifications may also increase the compounds' solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently, or in lower doses than with the unmodified compound.

### EXAMPLES

Materials. All reagents, unless specified, were purchased from Sigma-Aldrich.

Protein expression and purification. Proteins were cloned, expressed, and purified as described (J. Struct. Biol. 2012, 180, 551-562). His₁₂-PfAtg8^{CM} variants were expressed and purified with Cobalt-NTA affinity columns similar to His₆-PfAtg8^{CM}, as previously published with the exception that proteins were eluted from Cobalt-charged TALON resin (Clonetech) in buffer containing 50 mM EDTA rather than imidazole.

Surface Plasmon Resonance Assays. SPR runs were conducted on a Biacore 3000 instrument (GE Healthcare) at 25° C. with a flow rate of 50 μL/min, unless otherwise specified. Running buffer (RB) consisted of 1× PBS (1 mM KH₂PO₄, 5.6 mM Na₂HPO₄, 154.5 mM NaCl, pH 7.4), 0.01% v/v P20, and varying amounts of DMSO (Quality Biologicals). Binding and equilibrium constants were determined with Scrubber (BioLogic™). A double referencing method was applied to correct for nonspecific binding to the chip with interspersed blank injections correcting for baseline drifts. Changes in refractive index due to DMSO were accounted for with a DMSO calibration curve.

MMV SPR competition assay. MBP-PfAtg3 was immobilized onto a CM5 chip (Biacore) as described previously, with MBP immobilized on a reference flowcell. Compounds were added to 300 nM His₆-PfAtg8^{CM} in RB at a final concentration of 5 μM and 40 μL was injected, followed by a 12.5 μL injection of 2 M MgCl₂ for dissociation and regeneration of the SPR chip surface. The final DMSO concentration in SPR runs was 3%.

SPR dose-dependent inhibition. 30 μL of PfAtg8^{CM} at 200 nM was injected in the presence of a 2-fold dilution series of compound (highest concentration of 50 μM) or equivalent volume of DMSO (final DMSO concentration was 1%). Each injection was followed by a 10 μL injection of 2 M MgCl₂. All measurements were conducted in triplicate.

Direct binding of compound 1 to PfAtg8^{CM}. His₁₂-PfAtg8^{CM} was injected over an NTA-chip (GE Healthcare) preconditioned with nickel, leading to capture of 3,000 response units (RUs). Running buffer contained 3% DMSO. A 2-fold-dilution series of compounds, highest concentration of 75 μM, was injected over PfAtg8 variants at 40 μL/minute.

Direct binding of compound 8 to PfAtg8^{CM}. Compound 8 was injected over a neutravidin-coated SPR chip (GE Healthcare) on one flowcell, with 130 RUs immobilized, while BACH was injected over a reference flowcell (150 RUs immobilized). His₆-PfAtg8^{CM}, Human LC3, or His₆-PfAtg3 was injected in duplicate in running buffer containing 10 mM HEPES pH 7.5, 150 mM NaCl, 0.05% P20. Protein was dissociated from the chip after each cycle with 10 μL injection of 20 mM HEPES pH 7.4, 1% w/v SDS regeneration solution.

Synthesis of PTA derivatives. All reagents were obtained from commercial suppliers and used without further purification. Acetonitrile was distilled after drying on CaH₂ then stored over 3 Å molecular sieves. Yields of all reactions refer to the purified products. Dynamic Adsorbents 32-63 μm silica gel was used for flash column chromatography and 250 μm w/h F254 plates were used for thin layer chromatography (TLC). Microwave-assisted reactions were carried out using a Biotage Initiator Microwave Synthesizer (300 W). ¹H and ¹³C NMR spectra were acquired on a Bruker Avance III 500 spectrometer operating at 500 MHz for ¹H and 125 MHz for ¹³C. Chemical shift values are reported as δ (ppm) relative to CHCl₃ at δ 7.27 ppm and DMSO at δ 2.50 ppm for ¹H NMR and CHCl₃ at δ 77.0 ppm and DMSO at δ 39.51 ppm for ¹³C NMR. Mass spectrometry analysis was carried out at University of Illinois at Urbana-Champagne, School of Chemical Sciences, Mass Spectrometry Laboratory. The purity of synthesized compounds was ≧95% as analyzed by HPLC (Beckman Gold Nouveau System Gold) on a C₁₈ column (Grace Alltima 3 μm C₁₈ analytical Rocket® column, 53 mm×7 mm) using triethylammonium acetate buffer (50 mM, pH 7) and ACN as eluent, flow rate 3 mL/minute, and detection at 300 nm.

4-Formyl-N-[4-(pyridin-2-yl)-1,3-thiazol-2-yl]benzamide (Compound 7). To a solution of 4-(pyridin-2-yl)-1,3-thiazol-2-amine (0.059 g, 0.33 mmol) in acetonitrile (2.0 mL) was added sequentially dicyclohexylcarbodiimide (0.076 g, 0.37 mmol), 4-formylbenzoic acid (0.050 g, 0.33 mmol), and N,N-dimethylamino pyridine (0.012 g, 0.10 mmol). The mixture was heated at 50° C. for 17 hours, then allowed to cool to ambient temperature. Solids were removed by vacuum filtration, and the resulting filtrate was condensed under reduced pressure. The resulting yellow solid was redissolved in CHCl₃ (5 mL) and 1M HCl (5 mL) was added to give a yellow-tan emulsion at the liquid-liquid interface. This solid was collected by centrifugation at 4000 rpm for 5 minutes followed by manual collection of the resulting cake (this acid precipitation was necessary to remove closely eluting impurities). The solid was then purified by silica flash column chromatography (DCM:MeOH:Triethylamine 94:5:1) R_{f}=0.32. The product was obtained as a yellow powder (23 mg, 22% yield). ¹H NMR (500 MHz, DMSO-d₆) δ (ppm)=12.95 (br. s., 1H), 10.13 (s, 1H), 8.63 (d, J=3.93 Hz, 1H), 8.31 (d, J=8.17 Hz, 2H), 8.07 (d, J=8.33 Hz, 2H), 8.03 (d, J=7.86 Hz, 1H), 7.92 (s, 1H) 7.91 (td, J=2.00 Hz, 8.75 Hz, 1H), 7.35 (ddd, J=1.10, 4.79, 7.47 Hz, 1H) ¹³C NMR (500 MHz, DMSO-d₆) δ (ppm)=192.90, 164.74, 158.91, 152.03, 149.54, 149.38, 138.50, 137.30, 137.15, 129.41, 128.94, 122.88, 120.06, 112.30. HRMS (ESI) m/z: calc'd 310.0650 (M−H⁺); found 310.0651 (M−H⁺).

4-[[2-[6-[5-[(3aR,4R,6aS)-2-Oxo-1,3,3a,4,6,6a-hexahydrothieno[3,4-d]imidazol-4-yl]pentanoylamino]hexanoyl]hydrazinyl]methyl]-N-(4-pyridin-2-yl-1,3-thiazol-2-yl)benzamide (Compound 8). 25 μL of a 50 mM solution of compound 7 was dissolved in DMSO was incubated with 20 μL of 50 mM BACH (5-[(3aS,4S,6aR)-2-oxo-1,3,3a,4,6,6a-hexahydrothieno[3,4-d]imidazol-4-yl]-N-(6-hydrazinyl-6-oxohexyl)pentanamide) (Sigma-Aldrich) dissolved in DMSO and 10 μL sodium acetate (pH 4.5) with 0.02% sodium azide at 37° C. O/N. Final concentration of compound 8 was 18 mM.

4-(Dimethoxymethyl)-N-[4-(pyridin-2-yl)-1,3-thiazol-2-yl]benzamide (compound 9). Compound 7 dissolved in CHCl₃, was treated with 1M HCl as described above, to form the hydrochloride salt. Compound 7 HCl (0.040 g, 0.12 mmol) was suspended in MeOH (0.5 mL) and trimethylorthoformate (0.010 mL, 0.91 mmol) was added followed by p-toulene sulfonic acid monohydrate (0.003 g, 0.012 mmol). This solution was heated by microwave irradiation at 130° C. in a sealed vial for 5 minutes then stirred at ambient temperature for 36 hours at which time a precipitate formed. The solvent was removed under reduced pressure and the residue was dissolved in DCM (10 mL) and washed with saturated NaHCO₃ (10 mL) brine (10 mL), and dried with Na₂SO₄. Condensation under reduced pressure yielded the product as a yellow powder (26 mg, 61% yield). ¹H NMR (500 MHz, CDCl₃) δ (ppm)=9.81 (br. s., 1H), 8.64 (d, J=4.24 Hz, 1H), 7.96 (d, J=8.17 Hz, 2H), 7.91 (d, J=7.86 Hz, 1H), 7.74 (s, 1H), 7.74 (td, J=1.73, 7.70 Hz, 1H), 7.62 (d, J=8.17 Hz, 2H), 7.22 (dd, J=4.95, 6.84 Hz, 1H), 5.47 (s, 1H), 3.35 (s, 6H) ¹³C NMR (500 MHz, CDCl₃) δ (ppm)=164.24, 158.12, 152.24, 149.85, 149.62, 143.32, 136.84, 131.80, 127.51, 127.29, 122.69, 120.50, 112.21, 102.08, 52.71. HRMS (ESI) m/z: calc'd 356.1069 (M−H⁺); found 356.1070 (M−H⁺).

Thermal shift assays. Assays were conducted in 1× PBS with 1:1800 final dilution of SYPRO® orange dye (Invitrogen). Measurements made in triplicate for each condition. Concentration of His₁₂-PfAtg8^{CM} or His₆-PfAtg8^{CM} in assay was 65 μM. Fluorescence was measured from 20° C. to 80° C. in a Biorad C1000™ thermal cycler. 100 μM PTA compounds or equivalent volume of DMSO was added to His₆-PfAtg8^{CM}.

In vitro infection of human hepatocytes with P. falciparum 3D7-GFP. The HC-04 cell line (ATCC, Manassas, Va., USA) was maintained in complete medium (IMDM containing 2.5% FCS, 100 units/ml penicillin, 100 μg/ml streptomycin and 2 mM L-glutamine, all from GIBCO®, Life Technologies, Grand Island, N.Y.). P. falciparum 3D7-GFP parasite strain (PLoS ONE 2010, 5, e9156) was propagated in the Parasitology Core facility, the Johns Hopkins Malaria Research Institute. In vitro infection of human hepatocytes was done as described previously (PLoS ONE 2013, 8, e75321). Briefly, salivary glands were sequestered from infected Anopheles gambiae mosquitoes at day 17 after exposure to infective blood meal and homogenates were separated on an OptiPrep™ Density Gradient (Sigma-Aldrich, St. Louis, Mo.) at 12,000×g for 10 minutes. Sporozoites were collected from the gradient interface, washed in complete medium, counted using a hemocytometer and incubated with HC-04 cells at 3:1 sporozoite to hepatocyte ratio for 2 hours at 37° C. Infected cultures were further propagated in complete medium alone or medium supplemented with 3 μM or 30 μM of compound 1. Flow cytometry based detection of infected cells was done 72 hours post infection using FACSCalibur flow cytometer (BD Biosciences) and analyzed using FlowJo software (Tree Star, Inc., Ashland, Oreg.).

The effect of compound 1 on the viability of in vitro propagated HC-04 cells was monitored as following: 0.3×10⁶ cells per well were seeded into the 24-well plate and treated with 3 μM or 30 μM of compound 1 in complete medium for 96 hours, relevant amount of DMSO was used as a vehicle control. Detection of Annexin-V positive and PI-positive cells in hepatocyte cultures was done by flow cytometry according to the manufacturer's instruction (Invitrogen™, Life Technologies, Grand Island, N.Y., USA).

P. falciparum blood stage culturing. P. falciparum 3D7 and FCR3 cultures were maintained using modified, previously published methods at 37° C., 2% hematocrit of human red blood cells (Science 1976, 193, 673-675). Complete culture media consisted of sterile RPMI 1640 media (Life Technologies), supplemented with 10% human serum, 0.005% hypoxanthine, and buffered with final concentrations of 0.6% HEPES and 0.26% NaHCO₃. The FCR3 strain was maintained at 3% CO₂ and 5% O₂, 92% N₂ atmosphere, while the 3D7 strain was maintained at 5% CO₂, 5% O₂, 90% N₂ atmosphere.

Immunoblot analysis of P. falciparum blood stage. P. falciparum FCR3 (generously provided by Dr, J. Smith, Seattle BioMed) asynchronous culture, 25% parasitemia, was washed in starvation media lacking human serum and resuspended in complete media with 50 μM compound 1 or equivalent DMSO, or starvation media with equivalent DMSO for five hours. RBCs were harvested with centrifugation, lysed with 0.2% saponin, and RBC lysate was removed through three 1× PBS washes. Parasites were harvested by centrifugation and washed in 1× PBS with Complete EDTA-free protease inhibitors (Roche) and lysed by repeated vortexing and boiling in SDS reducing sample buffer. Lysates were separated with SDS-PAGE on a 15% polyacrylamide gel and subjected to western blotting with 1:400 α-TgAtg8, demonstrated to be cross-reactive with PfAtg8 (PLoS ONE 2013, 8, e79059) (generously provided by Dr. P. Roepe, Georgetown University). HRP-conjugated secondary antibodies (Southern Biotech) were detected by SuperSignal West Femto (Thermo Scientific) or Amersham™ ECL™ Prime (GE Healthcare) chemiluminescent substrate. AP-conjugated secondary antibodies (EMD Millipore) were detected using NBT/BCIP (Promega) colorimetric stain. Total protein levels were visualized with ProAct™ Membrane Stain (Amresco) and quantified with ImageJ. Parasite morphology at time of harvesting was visualized with light microscopy at 100× magnification on Olympus BX53 system microscope (Olympus America, Inc.).

SYBR Green I growth inhibition assay. 10 μL of 10× compound diluted in RPMI 1640 media (Gibco) with a constant concentration of 1% DMSO was added to a 96 well plate (Costar), 90 μL of 1.5% ring stage, synchronized with 5% w/v sorbitol P. falciparum 3D7 parasites, 1% hematocrit, in culture media with 10% v/v human serum with 10 μg/mL gentamycin. Each compound concentration and 1% v/v DMSO controls were run in triplicate. Plates were incubated at 37° C. in 5% O₂, 5% CO₂ and 90% N₂ for 72 hours. Plates were frozen, thawed, and incubated with 100-μL 2× SYBR green in lysis buffer (20 mM Tris pH 7.5, 5 mM EDTA, 0.008% Saponin, 0.08% TritonX-100) in the dark for at least one hour. Fluorescence was measured with a plate reader (HTS 7000, Perkin Elmer) at excitation/emission wavelengths of 485/535 nm.

In silico docking. Docking was conducted using the OpenEye software package using standard parameters if not specified otherwise (eyesopen.com) (J. Comput. Aided Mol. Des. 2012, 26, 897-906). A receptor for PfAtg8 was made with make_receptor from OEDocking toolkit without constraints covering the whole molecule to detect potential binding pockets on the surface. Three main pockets (W-site, L-site, A-site) were detected with 377, 271, and 513 Å³ volumes, used in first docking studies. A second receptor was generated with specific constraints to the carbonyl of Lys47 as hydrogen bond donor. A maximum of 2000 conformers for each compound from the MMV Malaria Box were prepared with Omega2 (J. Chem. Inf. Model. 2010, 50, 572-584; J. Chem. Inf. Model. 2012, 52, 2919-2936). FRED was used to dock these conformers onto both the constrained and unconstrained receptor. Docking results were visualized using the OpenEye visualization software, VIDA.

Generation of homology models for P. yoelii and P. berghei Atg8. Using iTasser, models of P. yoelii and P. berghei Atg8 were generated with PDB code 4EOY as the parent molecule (Nat. Protoc. 2010, 5, 725-738). Default values as suggested by the webserver were used to generate these models. Sequences for P. yoelii (PYYM_0504500) and P. berghei (PBANKA_050410) were obtained from PlasmoDB.

Human Subjects and Hazards. P. falciparum, a human pathogen, is cultivated in human erythrocytes. The organisms are maintained in licensed BSL2 facilities, and approvals are obtained annually for all consortia laboratories. Human erythrocytes are obtained either commercially or from healthy volunteers under Johns Hopkins IRB-approved protocols. Since these cells are provided to the lab without identifiers their use does not constitute human investigation.

### Experimental Methods for Compounds C5-C30

Bacterial strains and culture conditions. Escherichia coli strains were cultured in Terrific Broth (TB) medium supplemented with 50 μg/ml ampicillin or kanamycin as appropriate. All proteins were expressed using the E. coli Rosetta 2 strain, which has an additional plasmid for expressing rare codons with a chloramphenicol resistance cassette.

Plasmid and strain construction. The plasmids and strains used to express hLC3, PfAtg8 and PfAtg3 have been described previously (J Struct Biol 180:551-562 (2102)). hAtg3 was received from the Johns Hopkins Genetic Resources Core Facility on a pENTR221 vector. The gene was PCR amplified using custom primers 5′ CATGCCATGGGCCAGAATGTGATTAATACTGTGAAGGGAA (sense) (SEQ ID NO: 1) and 5′ CGCGGATCCGTTACATTGTGAAGTGTCTTGTGTAGTC (antisense) (SEQ ID NO: 2). The PCR fragment was digested with NcoI and BamHI restriction enzymes (New England BioLabs), and then ligated into a modified pRSF vector containing an N-terminal maltose binding protein (MBP) tag followed by a tobacco etch virus (TEV) protease cleavage site (J Mol Recognit 26:496-500 (2013)). The plasmid was then transformed into chemically competent E. coli Rosetta 2 cells.

Protein expression and purification. For hLC3, PfAtg8, and PfAtg3, expression and purification were performed as previously described (J Struct Biol 180:551-562 (2012)).

After transformation of hAtg3, a single colony was selected and sequence verified. Cells from this stock were grown in Terrific Broth (TB) media with 50 μg/mL kanamycin and 34 μg/mL chloramphenicol. Cells were grown in the presence of 1% glucose at 37° C., shaking at 250 rpm until an OD₆₀₀ of 3.0 was reached. At this point, the cells were induced with 0.5 mM IPTG and then continued growing overnight at 20° C. with shaking.

All steps after overnight incubation were conducted at 4° C. The cells were harvested by centrifugation at 1300 g for 45 min and resuspended in lysis buffer: 10 mM HEPES pH 7.5 150 mM NaCl, 1 mM DTT with one tablet of Roche® Complete EDTA-free protease inhibitor per 100 mL and benzonase (Sigma). Using an EmulsiFlex C5 cell disruptor, the cells were lysed at 15 kPSI (100 MPa). This lysed solution was cleared at 18,000 g for 45 min, after which the supernatant was decanted to incubate with amylose resin (NEB) for 30 min. Resin was washed with lysis buffer before the protein was incubated for 30 min with elution buffer (lysis buffer with 10 mM maltose). Eluted hAtg3 was cleaved with homemade Tobacco Etch Virus (TEV) protease at a mass ratio of 1:100, while simultaneously dialyzed into 10 mM HEPES pH 7.5, 100 mM NaCl, 1 mM DTT. After cleaving, hAtg3 was separated from MBP and TEV protease by anion exchange using a RESOURCE Q column (GE Healthcare) on an ÄKTA purifier system.

Purified proteins were analyzed by SDS-PAGE and quantified using a Nanodrop (Thermo Fisher) spectrophotometer at λ=280 nm using the molar extinction coefficient for each of the proteins as calculated from the primary sequence by ProtParam (Gasteiger E, et al., Protein identification and analysis tools on the ExPASy server, p 571-607, The proteomics protocols handbook Springer (2005)). For surface plasmon resonance (SPR), proteins were concentrated to 1-3 mg/ml and buffer exchanged into the indicated running buffer.

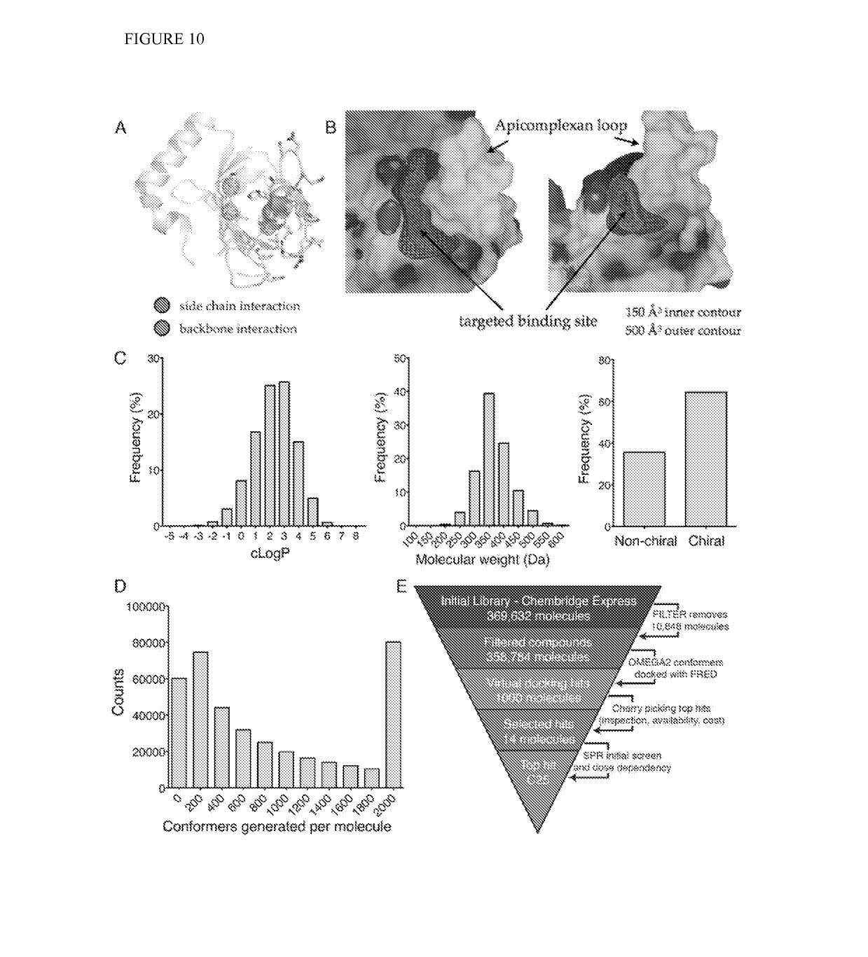

Virtual Ligand Screening (VLS) by FRED. The OpenEye suite of programs (OpenEye Scientific Software Inc. 2012. OEChem, Version 1.8.1, Santa Fe, N. Mex., USA) was utilized to prepare an in silico library of the ChemBridge commercial small molecule database. Structure description files (sdf) were obtained from ChemBridge and first subjected to the OpenEye FILTER program for removal of known toxic and reactive groups. Next a conformer library was generated using OMEGA2 (J Chem Inf Model 50:572-584 (2010)) with a maximum of 2000 conformers per small molecule and otherwise using default parameters. This conformer library was then docked against the co-crystal structure of PfAtg8-PfAtg3, PDB code 4EOY, after removing the PfAtg3-peptide and water molecules. The protein-receptor was generated with OpenEye's make_receptor program using default values and adding two backbone interaction restraints located in the apicomplexan loop pocket as anchoring points (FIGS. 10A,B). Docking was carried out on a 24 core MacPro using FRED (Comput Aided Mol Des 26:897-906 (2012)) with default settings using a fine grid of 0.5 Å. Visualization and inspection of the top 500 docking results was carried out with OpenEye's visualization software, VIDA. A selection of 29 molecules were cherry picked, from which 14 were readily available from commercial sources for testing in biochemical and biophysical assays (Table 1).

TABLE 1
Small molecule probes of the apicomplexan loop pocket.

| Com- | | | | | PubChem |
|---|---|---|---|---|---|
| pound | IUPAC | CID | Structure | Mol Wt | XLogPI |
| C5 | 5-nitro-N-(pyridin-3- ylmethyl)furan-2- carboxamide | 633996 | 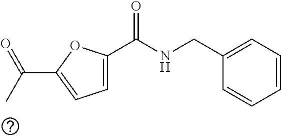 [SMILES: CC(=O)c1ccc(C(=O)NCc2ccccc2)o1] | 242.21 | 1.2 |
| C11 | N-([7-(pyridin-4-yl)- 2,3-dihydro-1- benzofuran-2-yl] methyl)-4,5,6,7- tetrahydro-1-benzo- thiophene-1- carboxamide | 45176114 | 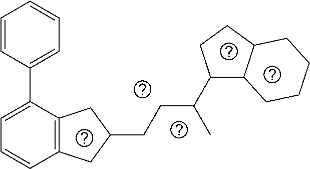 [SMILES: CC(CCC1Cc2cccc(-c3ccccc3)c2C1)C1CCC2CCCCC21] | 390.50 | 4.4 |
| C13 | 1-(4-bromobenzene- sulfonyl)-N-(furan- 2-ylmethyl)piperidine- 4-carboxamide | 1327632 | 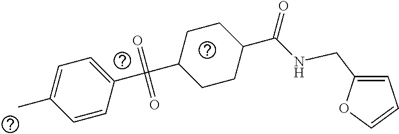 | 427.31 | 2.3 |
| C16 | N-[(3-ethyl-1H- pyrazol-4-yl)methyl]- 5-phenyl-1H-pyrrole- 2-carboxamide | 54761948 | 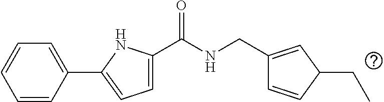 [SMILES: CCC1C=CC(CNC(=O)c2ccc(-c3ccccc3)[nH]2)=C1] | 294.35 | 2.3 |
| C17 | N-[2-((6-ethyl-1- methyl-1H- pyrazolo[3,4-a] pyrimidin-4-yl)amino) ethyl]pyridin-3-amine | 58834679 | 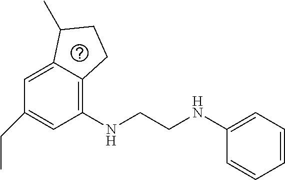 [SMILES: CCc1cc(NCCNc2ccccc2)c2c(c1)C(C)CC2] | 297.36 | 1.8 |
| C18 | N-[2-(N-methyl- methanesulfonamide) ethyl]-5-phenyl- 1H-pyrrole-2- carboxamide | 56745406 | 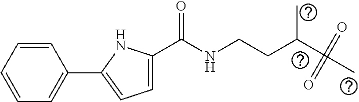 | 321.40 | 1.3 |
| C19 | 3-ethyl-N-([2- (pyrazin-2-yl)-1,3- thiazol-4-yl]methyl)- 1H-indole-2- carboxamide | 58966056 | 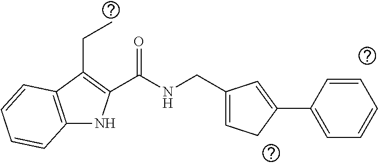 [SMILES: CCc1c(C(=O)NCC2=CCC(c3ccccc3)=C2)[nH]c2ccccc12] | 363.44 | 2.6 |
| C20 | 5-(2-chlorophenyl)- 2-methyl-N-[(6- oxo-3,6-dihydro- pyrimidin-4-yl) methyl]furan-3- carboxamide | 58965334 | 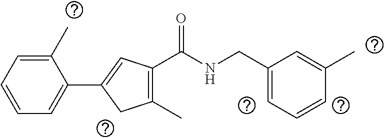 [SMILES: CC1=C(C(=O)NCc2cccc(C)c2)C=C(c2ccccc2C)C1] | 343.76 | 1.8 |
| C22 | 2-ethyl-4-methyl- N-([2-(pyrimidin- 2-yl)-1,3-thiazol- 4-yl]methyl)-1,3- thiazole-5- carboxamide | 60974374 | 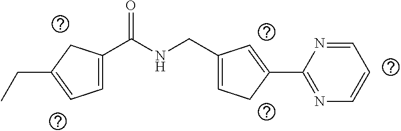 [SMILES: CCC1=CC=C(C(=O)NCC2=CCC(c3ncccn3)=C2)C1] | 345.44 | 0.8 |
| C24 | N-([1-(1H-imidazol- 5-ylmethyl) pyrrolidin-3-yl] methyl)-8-methyl- imidazo[3,2-a] pyridine-2- carboxamide | 56723608 | 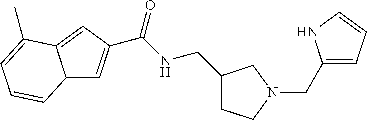 [SMILES: CC1=CC=CC2C=C(C(=O)NCC3CCN(Cc4ccc[nH]4)C3)C=C12] | 338.41 | 1.8 |
| C25 | 1-[(3-methylphenyl) methyl]-3-([1- pyridin-2-ylmethyl- pyrrolidin-3-yl] methyl)urea | 56758287 | 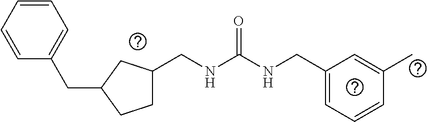 [SMILES: Cc1cccc(CNC(=O)NCC2CCC(Cc3ccccc3)C2)c1] | 338.45 | 2.1 |
| C28 | N-([2-(dimethylamino) pyridin-3-yl] methyl)-4-phenyl-1H- imidazole-5- carboxamide | 56749587 | 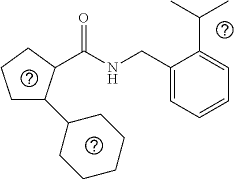 [SMILES: CC(C)c1ccccc1CNC(=O)C1CCCC1C1CCCCC1] | 323.38 | 3.2 |
| C29 | 5-(1-cyclopentyl- pyrrolidin-3-yl)-N- [(6-methyl-4-oxo- 1,4-dihydro- pyrimidin-2-yl) methyl]thiophene-2- carboxamide | 45208702 | 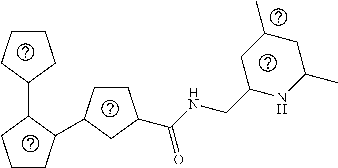 [SMILES: CC1CC(C)NC(CNC(=O)C2CCC(C3CCCC3C3CCCC3)C2)C1] | 386.51 | 2.3 |
| C30 | N-([3-(pyrazin-3-yl)- 1,3-thiazol-4- yl]methyl)-4H- thieno[3,2-b]pyrrole- 5-carboxamide | 50952642 | 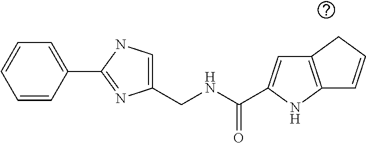 [SMILES: O=C(NCc1c[nH]c(-c2ccccc2)n1)c1cc2c([nH]1)C=CC2] | 341.41 | 1.6 |

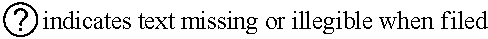

Surface Plasmon Resonance (SPR) protein-protein interaction assay. All measurements were carried out on a BiaCore 3000 instrument. We used the previously described SPR competition assay to test inhibition of the PfAtg8-PfAtg3 and hLC3-hAtg3 interaction. The standard running buffer was 10 mM HEPES, 150 mM NaCl, 0.01% Tween 20, adjusted to pH 7.5. Prior to performing SPR assays, all solutions were sterile filtered and degassed for 1 h at room temperature. For small molecule inhibitor studies, the same running buffer was supplemented with 1% DMSO. All dose dependency measurements were carried out in triplicate and corrected for DMSO absorption effects using double referencing with interspersed blank injections and an untreated flow cell on the SPR chip and the SPR analysis software Scrubber 2.0 (BioLogics). For each interaction pair, a new CM5 SPR chip was used. To exclude surface decay during the experiment, positive control injections were interspersed every 12^{th} injection. For all interaction studies, the Atg3 homologue was immobilized and the corresponding Atg8 partner was passed as an analyte over the chip in either the presence or absence of small molecule inhibitors, dissolved to 100 mM in 100% DMSO and tested at 500 μM final concentration. Prior to the immobilization of Atg3 to an SPR chip, a pH scouting from pH 6.0 to pH 4.0 in intervals of 0.5 pH units was carried out to determine the best pre-concentration conditions for immobilization. Briefly, flow cells of an SPR chip were activated using N-hydroxysuccinimide (NHS) and 1-ethyl-3-(3-dimethylaminopropyl)carbodiimide (EDC) for 7 min with a flow rate of 20 μl/min. For PfAtg3 immobilization, a solution of 50 μg/ml of PfAtg3 in 10 mM sodium acetate pH 5.0 was passed over one flow cell until >500 Response Units (RU) were captured. The same procedure was followed for the immobilization of hAtg3. Activated free binding sites on the chip were blocked using 1 M ethanolamine for an additional 7 min. All experiments were carried out at 25° C. with a 40 μl/min flow rate and 75 s contact time, followed by a 1 min dissociation time and a 25 μl regeneration injection of 2 M MgCl₂.

The dose-response curves were used to determine the approximate concentration of analyte (PfAtg8 or hLC3) needed to obtain a particular response unit (RU), typically in the range of 100-150 RU's. A fixed amount of analyte was then passed over the chip resulting in at least 100-150 response units (RU) in the absence of small molecules.

Two Species Inhibitor/Stabilizer (TSIS) Plot. To determine which inhibitors to pursue after the initial screen, it is useful not only to consider the variance of the control response (to determine if the inhibited response differs significantly from the control) but also to consider the inherent variability in the non-control responses, which is a function of many features, including the density of active protein on the chip. To do this, we considered the standard deviation of the non-control responses as a means by which to detect compounds that perform significantly better or worse than the majority. In this case, we pursued an inhibitor that performed significantly better at inhibiting the Plasmodium interaction while not interfering with the human interaction. We compared both responses normalized to their respective controls, with human plotted on the x-axis and P. falciparum on the y-axis. Each point corresponds to the average response in the presence of one compound, with error bars corresponding to the standard deviation in the individual measurements. The black rectangle in the graph encloses responses that are within one standard deviation of the average non-control response.

Thermal shift assay. Small molecules identified from our VLS approach were tested in thermal stability assays in 1× PBS with 1:1800 final dilution of SYPRO® orange dye (Invitrogen), with 65 μM His₆-PfAtg8^{CM}. Triplicate measurements of fluorescence were measured from 20° C. to 80° C. in a Biorad C1000™ thermal cycler for each concentration. C25 was added at increasing concentrations ranging from 100 μM to 2 mM, with DMSO concentration kept constant in all samples.

Plasmodium parasite growth inhibition assay. P. falciparum 3D7 parasites were cultured as previously described (J Med Chem 57:4521-4531(2014)). Briefly, the parasites were cultured in 10% v/v human serum with 10 μg/mL gentamycin and synchronized with 5% w/v sorbitol. 1.5% ring stage parasites were added to 96 well plates (Costar) containing serial dilutions, performed in triplicate, of compound in RPMI 1640 media (Gibco). Plates were incubated at 37° C. in 5% O₂, 5% CO₂, and 90% N₂ for 72 h. Plates were frozen, thawed, and incubated with 100 μL 2× SYBR green in lysis buffer (20 mM Tris pH 7.5, 5 mM EDTA, 0.008% Saponin, 0.08% TritonX-100) in the dark for 1 h. Fluorescence was measured with a plate reader (HTS 7000, PerkinElmer) at excitation/emission wavelengths of 485/535 nm.

Detection of P. falciparum exoerythrocytic forms in vitro. HC-04 human hepatocyte cell line (ATCC, Manassas, Va., USA) was maintained in complete medium (IMDM containing 2.5% FCS, 100 units/ml penicillin, 100 μg/ml streptomycin and 2 mM L-glutamine, all from GIBCO®, Life Technologies, Grand Island, N.Y.). P. falciparum 3D7HT-GFP parasite strain (PLoS One. 5(2):e9156. doi:10.1371/journal.pone.0009156 (2010)) was propagated in the Parasitology Core facility of the Johns Hopkins Malaria Research Institute. In vitro infection of human hepatocytes, and detection and quantification of P. falciparum 3D7HT-GFP EEFs by flow cytometry were performed as previously described (PLoS One. 8(9):e75321. doi:10.1371/journal.pone.0075321 (2013)).

Hepatocyte cytotoxicity assay. Hepatocyte cytotoxicity assays were carried out in triplicate at two concentrations of inhibitory compounds using HC-04 cells as previously described. Flow cytometry was used to assess two cytotoxicity markers via staining, namely propidium iodine (PI) and Annexin-V.

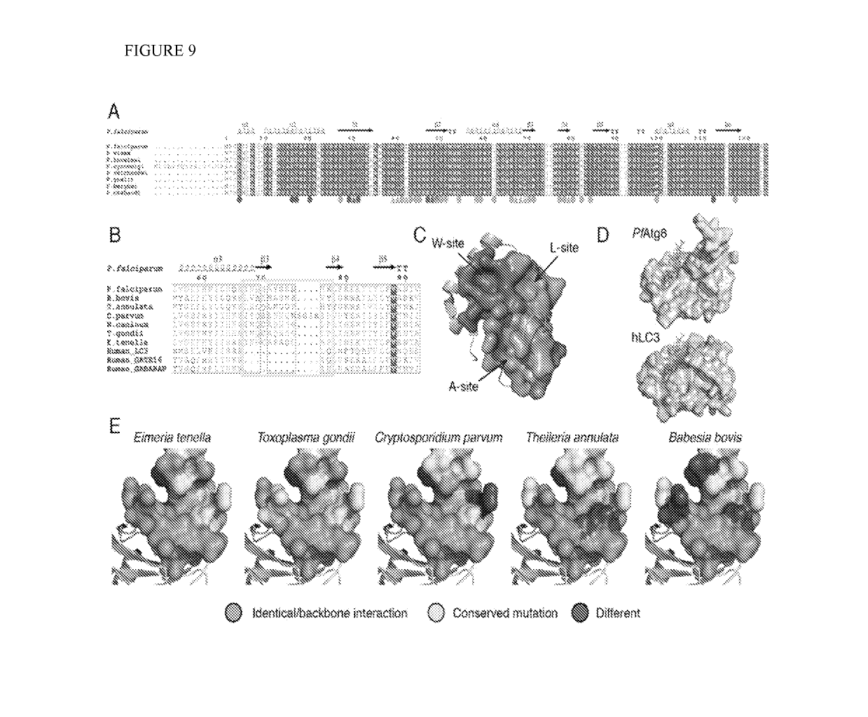

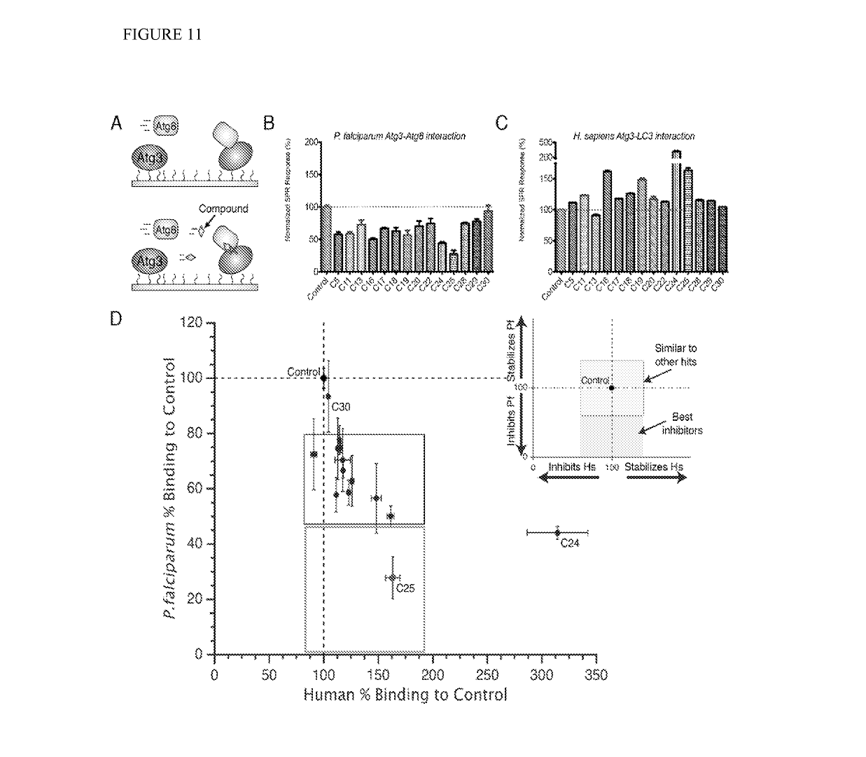

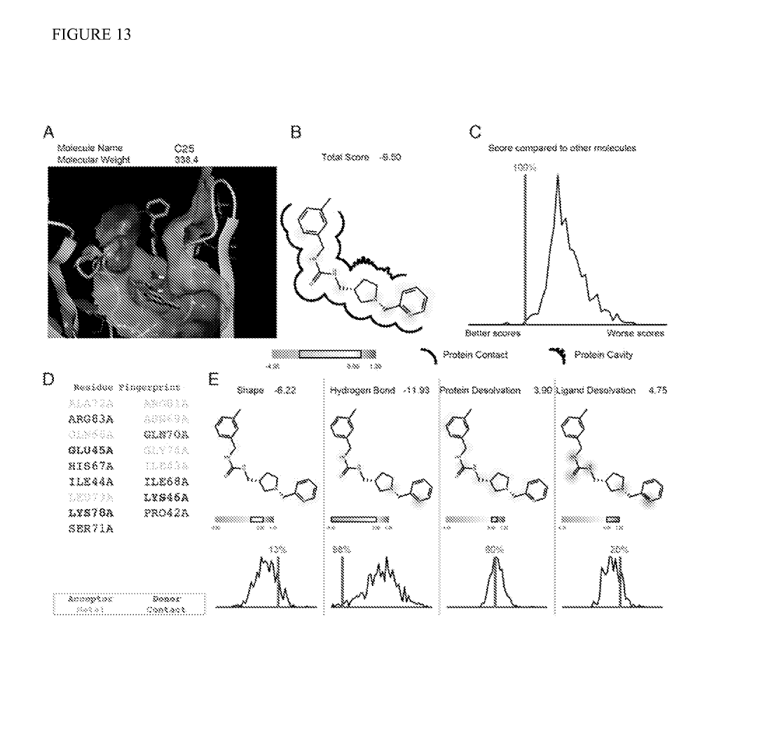

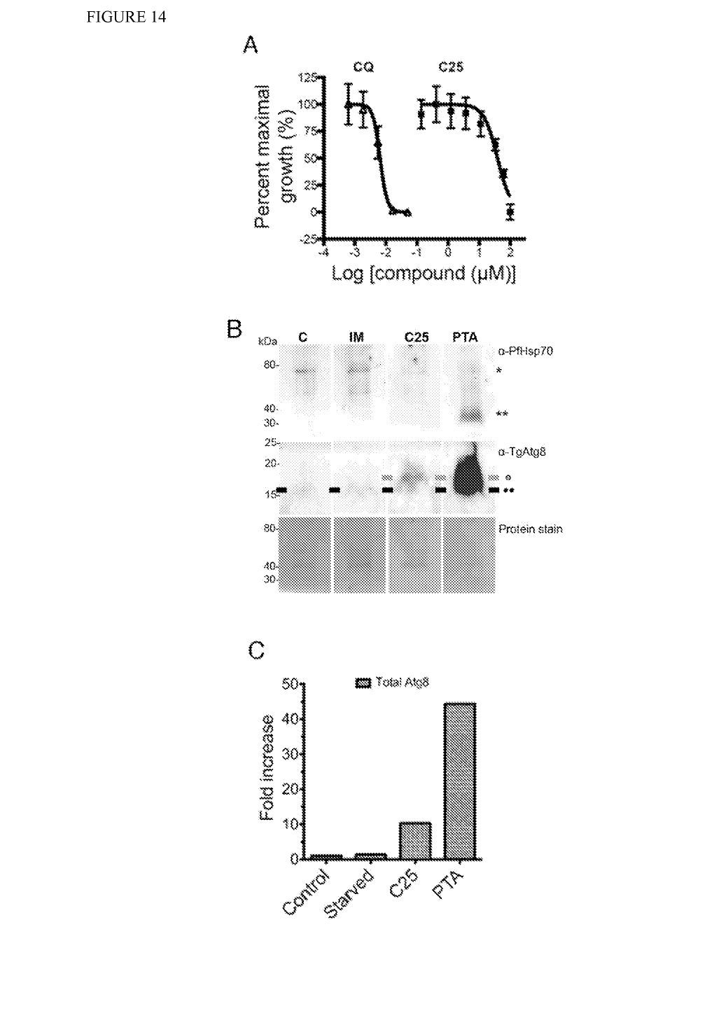

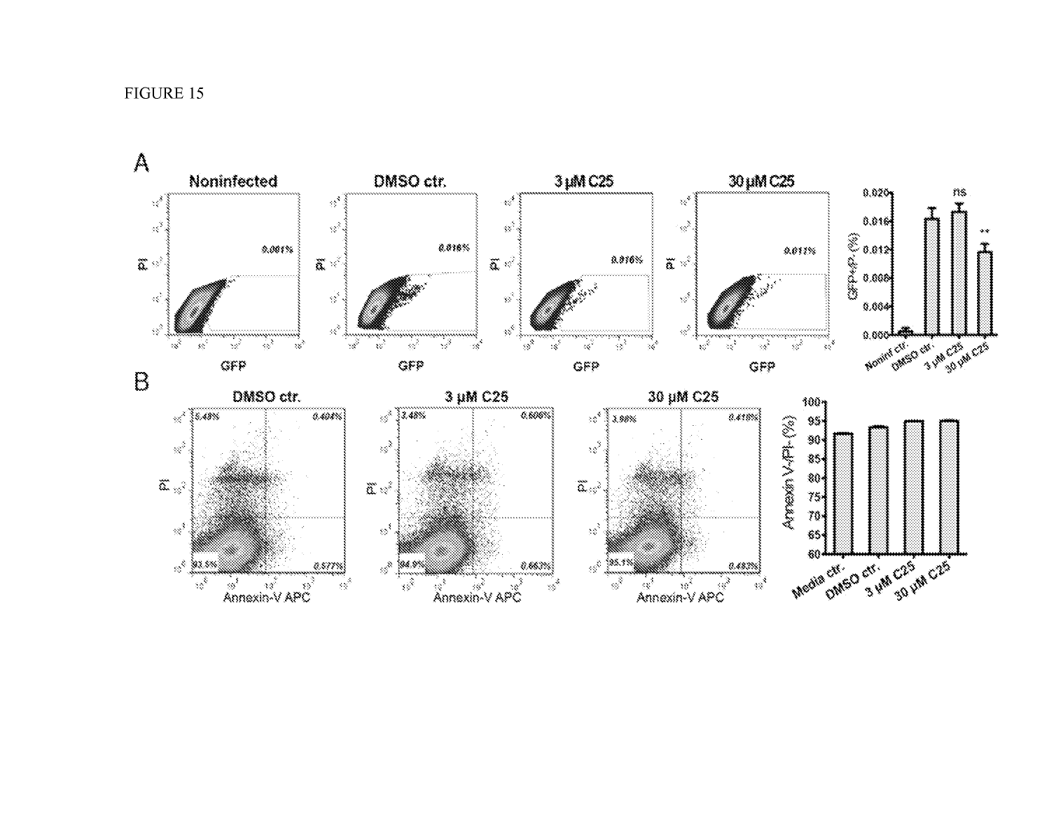

Web servers and software. Amino acid sequences of apicomplexan Atg8 proteins were retrieved from EuPathDB (Nucleic Acids Res 41:D684-691 (2013)) using the NCBI BLAST similarity search function (Nucleic Acids Res 41:W597-600 (2013)) to find proteins homologous to PfAtg8. Sequences of human Atg8 homologues were retrieved from UniProt (Nucleic Acids Res 43:D204-212 (2015)). Sequence alignments (FIGS. 9A,B) were performed using the ESPript multiple sequence alignment web server (espript.ibcp.fr) (Nucleic Acids Res 42:W320-324 (2014)). Clustal Omega was also used in sequence comparison (FIG. 9E) (Methods Mol Biol 1079:105-116 (2014)). FlowJo software was used in the analysis of flow cytometry data (FIGS. 15A,C) (Tree Star Inc. 2014. FlowJo, Version 10.0.6, Ashland, Oreg., USA). The plot for FIG. 11D was generated using Matlab (The Mathworks Inc. 2014. MATLAB, Version 8.4.0 (R2014b), Natick, Mass., USA.), and all bar graphs and concentration curves were generated using GraphPad Prism (GraphPad Software. 2007. Prism, Version 5.0a, La Jolla, Calif., USA.). Western blot quantification was performed with ImageJ (FIG. 14B) (ImageJ. U.S. National Institutes of Health, Bethesda, Md., USA). FIG. 10B was produced with OpenEye VIDA and rendered with POV-Ray (Persistence of Vision Pty. Ltd. 2004. Persistence of Vision™ Raytracer, Version 3.6, Williamstown, Victoria, Australia), FIG. 13A was produced with OpenEye VIDA, and all other molecular graphics were created with PyMOL (Schrodinger, LLC. 2010. The PyMOL Molecular Graphics System, Version 1.7, New York, N.Y., USA). Table 1 structures were produced with ChemBioDraw (Cambridge Soft Corporation. 2013. ChemBioDraw Ultra, Version 13.0.2.3020, Cambridge, Mass., USA) and X Log P3 values were retrieved from the PubChem compound database (Annual reports in Computational Chemistry 4:217-241 (2008)).

### Example 1

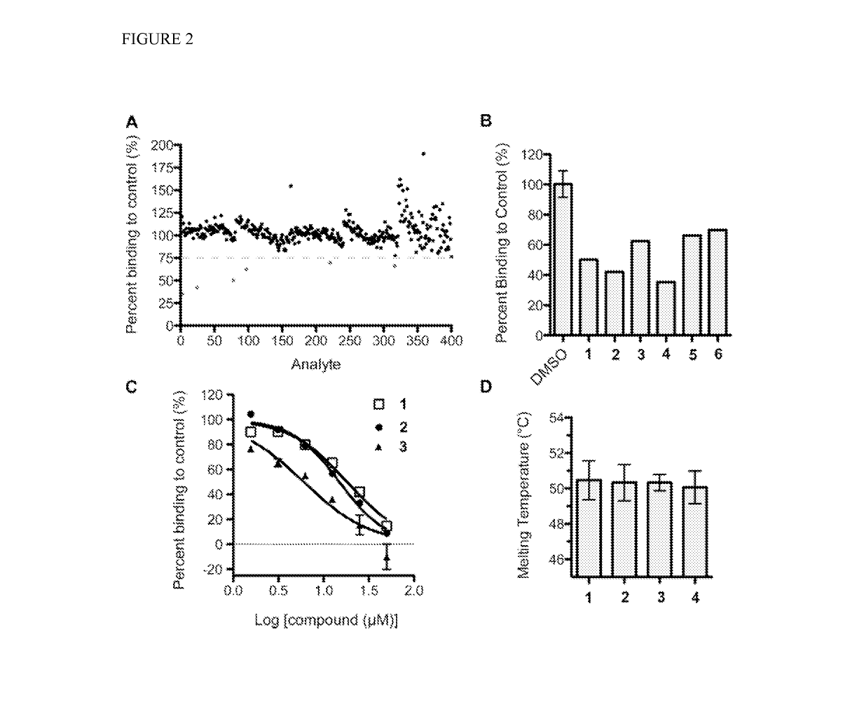

Screening of the MMV Malaria Box library. Previously, the inventors developed a surface plasmon resonance (SPR)-based competition assay to identify compounds that disrupt the PfAtg8-PfAtg3 noncovalent interaction. PfAtg3 is immobilized onto an SPR chip and PfAtg8 is injected in the presence of DMSO (control) or a compound (dissolved in DMSO) and binding is measured by the SPR response. The Medicines for Malaria Venture (MMV) Malaria Box of 200 drug-like and 200 probe-like molecules (PLoS ONE 2013, 8, e62906) was screened at 5 μM in a primary SPR screen (FIG. 2A). Six compounds met the cutoff for at least twenty five percent inhibition of the PfAtg8-PfAtg3 interaction: (N-(4-methylphenyl)-4-pyridin-2-yl-1,3-thiazol-2-amine) (compound 1), (2-methylsulfanyl-N-(4-pyridin-2-yl-1,3-thiazol-2-yl)benzamide) (compound 2), (2-bromo-N-(4-pyridin-2-yl-1,3-thiazol-2-yl)benzamide) (compound 3), N-[2-chloro-5-(trifluoromethyl)phenyl]-2-[2-(4-methylphenyl)pyrazolo[1,5-a]pyrazin-4-yl]sulfanylacetamide (compound 4), 1-[4-(dimethylamino)phenyl]-6,6-dimethyl-1,3,5-triazine-2,4-diamine (compound 5), 2-N,3-N-bis(4-bromophenyl)quinoxaline-2,3-diamine (compound 6) (FIG. 2B, Table 2).

In subsequent dose-dependent studies, compounds 4-6 demonstrated a constant level of inhibition independent of concentration of the small molecule and were not further investigated. Compounds 1-3 led to dose-dependent inhibition with an SPR inhibitory concentration (IC_{50 SPR}) ranging from six to 18 μM (FIG. 2C). Interestingly, these compounds shared a common scaffold: 4-pyridin-2-yl-1,3-thiazol-2-amine (abbreviated here as PTA), incorporated as the N-substituent in various anilines or benzamides. Compounds 1-3 were tested for their effect on the stability of PfAtg8^{CM} using fluorescence-based thermal shift assays (TSAs). None of the compounds significantly affected the melting temperature (Tₘ) indicating that they most likely did not disturb the tertiary structure of PfAtg8 (FIG. 2D).

TABLE 2
Structures and inhibition data for PTA compounds^{a}

| | CID | | | IC₅₀ (μM) (LEAN) | |
|---|---|---|---|---|---|
| Compound | (MMV ID) | Structure | Mol wt | SPR | Blood stage |
| 1 | 746602 (MMV007907) | 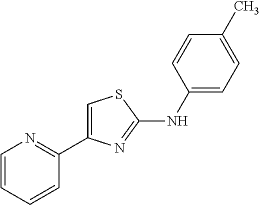 [SMILES: Cc1ccc(Nc2nc(-c3ccccn3)cs2)cc1] | 267.34 | 18.36 (0.25) | 1.47 (0.31) |
| 2 | 2526358 (MMV081245) | 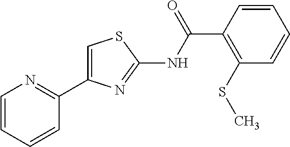 [SMILES: CSc1ccccc1C(=O)Nc1nc(-c2ccccn2)cs1] | 327.42 | 14.95 (0.22) | 0.20* (0.30) |
| 3 | 2454286 (MMV685809) | 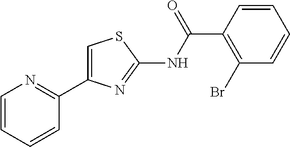 [SMILES: O=C(Nc1nc(-c2ccccn2)cs1)c1ccccc1Br] | 360.23 | 6.08 (8.28) | 1.58* (0.35) |
| 4 | 27668345 (MMV388660) | 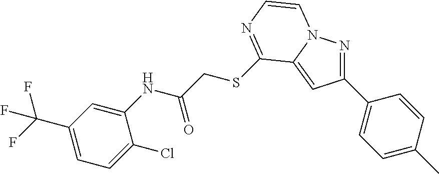 [SMILES: Cc1ccc(-c2cc3c(SCC(=O)Nc4cc(C(F)(F)F)ccc4Cl)nccn3n2)cc1] | 476.90 | None | Not tested |
| 5 | 427456 (MMV867482) | 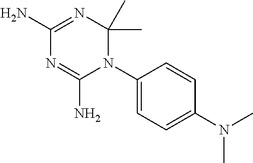 [SMILES: CN(C)c1ccc(N2C(N)=NC(N)=NC2(C)C)cc1] | 280.34 | None | Not tested |
| 6 | 1579827 (MMV007224) | 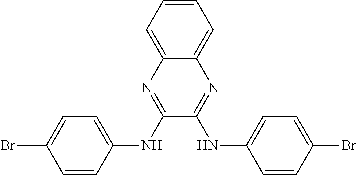 [SMILES: Brc1ccc(Nc2nc3ccccc3nc2Nc2ccc(Br)cc2)cc1] | 470.16 | None | Not tested |
| 7 | N/A | 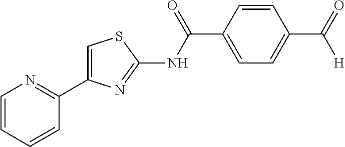 [SMILES: O=Cc1ccc(C(=O)Nc2nc(-c3ccccn3)cs2)cc1] | 309.34 | Not tested | |
| 8 | N/A | 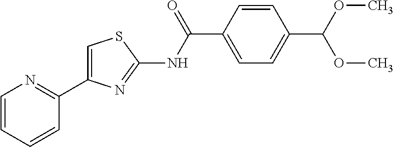 [SMILES: COC(OC)c1ccc(C(=O)Nc2nc(-c3ccccn3)cs2)cc1] | 355.41 | 2.66 (0.22) | 1.48 (0.23) |

^{a}PTA-containing compounds used in studies listed with PubChem Compound Identification (CID), MMV ID, chemical structure, molecular weight (g/mol), IC₅₀ in SPR and blood stage assays, and LEAN score, calculated as −log (IC₅₀)/number of heavy atoms.
Asterisks denote IC₅₀ values derived from the literature.

### Example 2

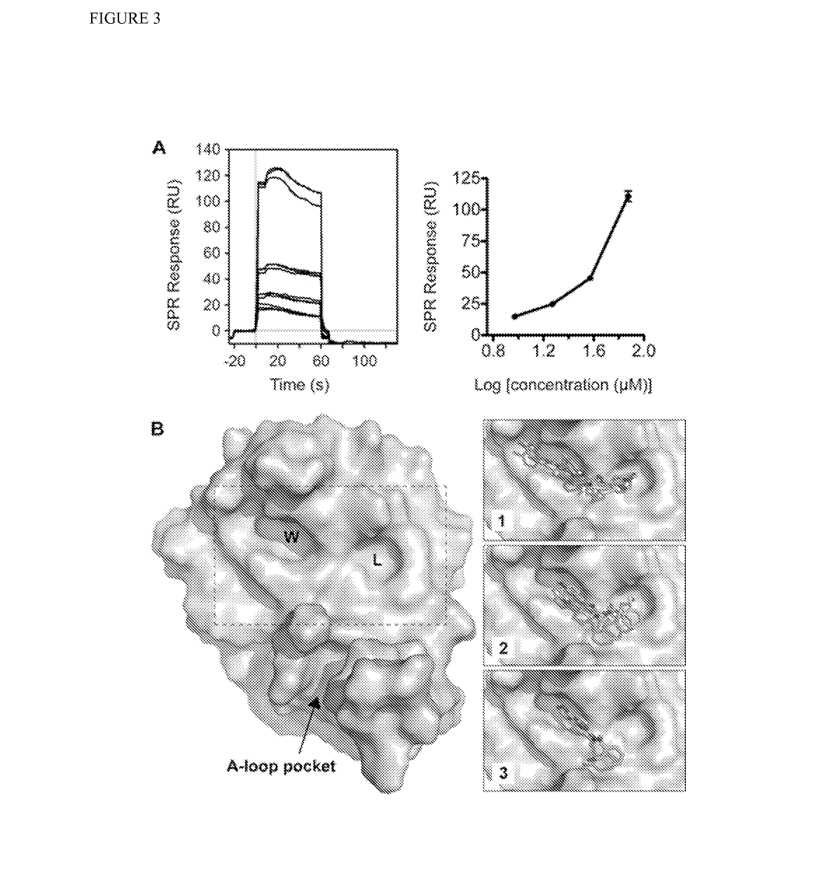

Determination of the PTA binding site on PfAtg8. The inventors tested whether these compounds bound directly to PfAtg8 using SPR. PfAtg8^{CM} was immobilized onto an SPR Ni-NTA chip via a twelve histidine N-terminal tag. Compound 1, chosen for its better solubility, was injected over the chip and binding was measured. Compound 1 led to a dose-dependent increase in SPR response, indicating binding to PfAtg8^{CM} (FIG. 3A). It was then sought to determine the binding site for the PTA compounds with in silico docking.

The OpenEye software package was used to dock conformers of the compounds against the X-ray structure of PfAtg8 (PDB code 4EOY). All three compounds docked to the W-site of PfAtg8 (FIG. 3B). Compounds 2 and 3 bound with the pyridine ring in the W-site, whereas compound 1 was predicted to bind with the pyridine ring in the L-site, the thiazole ring positioned between the pockets, and the methylbenzene group in the W-site. An alternate pose was enriched within the top ten poses output by the docking study, in which compound 1 binds solely within the W-site in a more compact conformation. It is worth mentioning that compound 1 is missing a donor oxygen compared to compounds 2 and 3, and therefore may adopt a different mode of binding to the W- and L-sites of PfAtg8. Compound 2 docked with the pyridine ring in the W-site and the methylsulfanylbenzene group bound to the L-site of PfAtg8. Docking was repeated using a version of the receptor for which the carbonyl of Lys47 was input as a hydrogen bond acceptor constraint (FIG. 3B inset). Compound 3 had the same docking pose in both studies. Compound 1 had a similar binding pose to the highest ranked pose from the unconstrained docking, while compound 2 was slightly different, with the pyridine ring rotated 90° in the W-site and the benzene ring positioned just below and to the left of the L-site pocket, with the methylsulfanyl group reaching into the mostly hydrophobic L-site.

### Example 3

Compound 1 shows activity against Plasmodium liver stages. The half maximal inhibitory concentrations (IC₅₀) for these compounds in P. falciparum 3D7 blood stages are previously reported and located on the NCBI PubChem database (pubchem.ncbi.nlm.nih.gov). Compound 1 has a reported IC₅₀ of 350 to 400 nM (PubChem bioassay ID (AID): 660866 and 449703). The reported IC₅₀ for compound 2 ranged from 0.20 to 6.8 μM while 3 compound ranged from 1.36 to 4.52 μM (PubChem AID: 660866 and 449707). Compound 1 was focused on for further studies because the reported cytoxicity in human cell lines is much lower than that of compounds 2 or 3 (PubChem AID: 660872, 685525, and 449705).

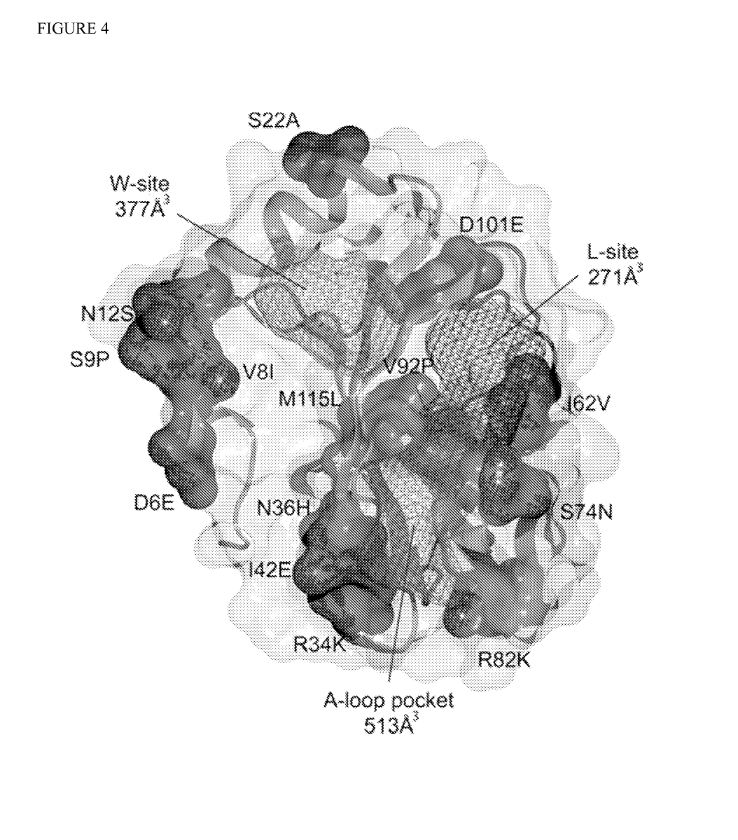

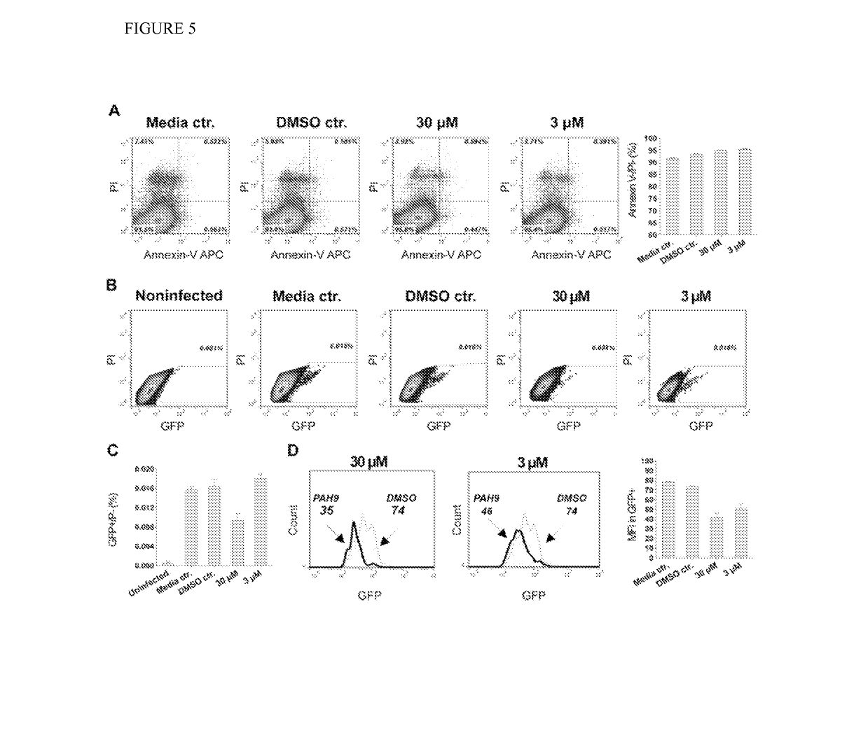

PfAtg8 is expressed and lipidated during the liver stage where it partially localizes to the apicoplast. Treatment of early liver stage parasites with the autophagy inhibitor 3-methyladenine is reported to delay conversion of the parasite into its trophozoite form. It was therefore hypothesized that compound 1 would also have activity against the liver stage. Compound 1 was previously tested in P. yoelii liver stage cultures and did not display >50% inhibition at the screening concentration of 10 μM; an IC₅₀ was not reported (PubChem AID: 602118 and 602156). P. yoelii and P. berghei are often used to test drugs for liver stage inhibition as they are easier to culture. However, these are rodent malaria models and may not be indicative of activity in P. falciparum. Analysis of the W/L-site in these three species revealed differences in the amino acid composition that could affect drugs predicted to bind in that region (FIG. 4). A recently established P. falciparum liver stage in vitro model in which sporozoites isolated from infected mosquitos' salivary glands was utilized to invade HC-04 hepatocytes (PLoS ONE 2013, 8, e75321). HC-04 is a unique immortalized cell line that exhibits the expression of biochemical markers characteristic for normal hepatocytes and allows for the full development of the human malaria parasite, P. falciparum. Using this system, the effect of compound 1 was assessed on the development of P. falciparum 3D7-GFP parasites in human hepatocytes in vitro. Though no change in the viability of HC-04 cells was detected in response to treatment with 3 μM or 30 μM of compound 1 for 96 hours (FIG. 5A). Using flow cytometry about a 50% decrease in the proportion of hepatocytes infected with P. falciparum 3D7-GFP sporozoites (GFP+/PI− cells) was observed in response to treatment with 30 μM, but not 3 μM of compound 1 (FIG. 5B, C). Additionally, there was a dose-dependent reduction in the intensity of GFP fluorescence at both concentrations of compound 1, indicating inhibition of parasite development within hepatocytes, at least in vitro (FIG. 5D). Because compound 1 did not affect cell survival or cell growth of HC-04 cells (FIG. 5A), the compound's effect on the parasite is unlikely to result from the host cell cytotoxicity.

### Example 4

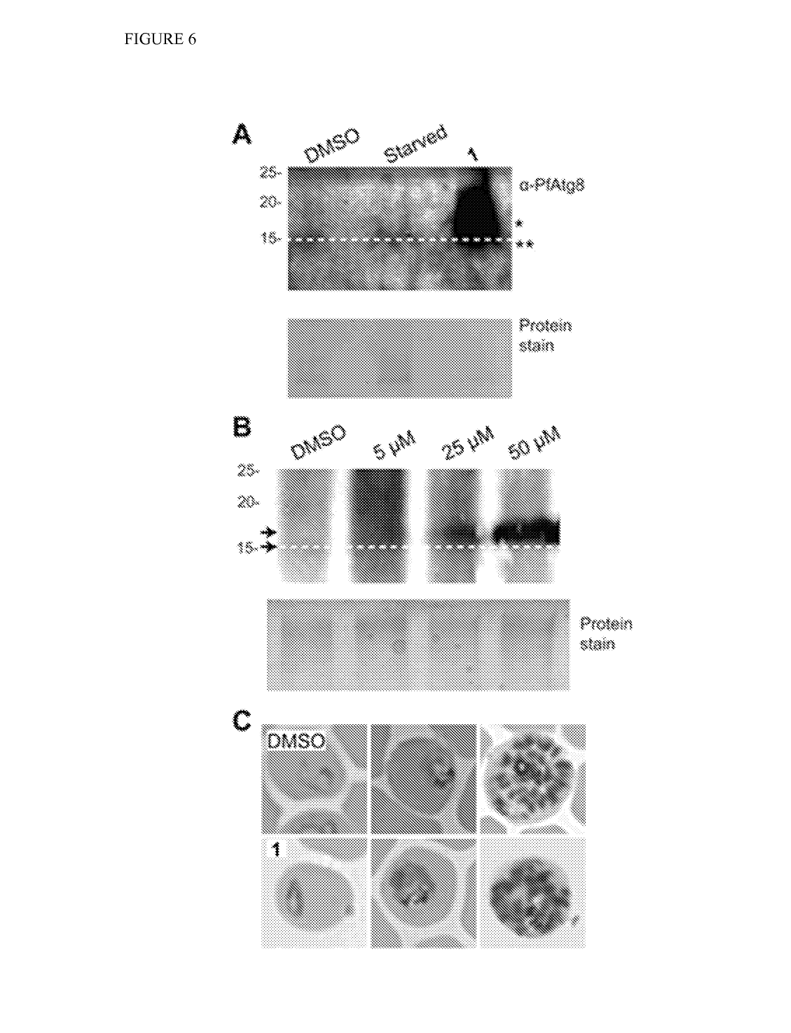

Validation of drug effect on PfAtg8 in parasite cultures. It was next sought to determine whether compound 1 had an effect on PfAtg8 in P. falciparum blood stage cultures. In immunoblot assays, very low levels of endogenous PfAtg8 were detected in DMSO-treated control cells. Incubation of cells in minimal media lacking human serum for five hours led to a very slight increase in PfAtg8. In contrast, treatment with 50 μM cytocidal levels of compound 1 led to a drastic increase in PfAtg8 protein levels as well as a shift in mobility, likely corresponding to the unlipidated form of PfAtg8. Overall protein levels were unchanged, indicating an up-regulation or accumulation of PfAtg8 in the presence of compound 1 under conditions that are likely leading to cell death (FIG. 6A). A dose-dependent decrease in lipidation of PfAtg8 is observed already at 5 μM of compound 1, and at 25 μM and 50 μM, the unlipidated PfAtg8 is exclusively detected (FIG. 6B) after treating parasites for 12 hours. When investigating the soluble versus insoluble membrane fraction, PfAtg8 could only be detected in the soluble fraction with the number of parasites used per lane. This was attributed to the low amount of PfAtg8 present in the untreated control and reaching the detection limit of our assay (data not shown). At the treatment concentration and duration used in the study, parasite morphology appeared normal (FIG. 6C).

### Example 5

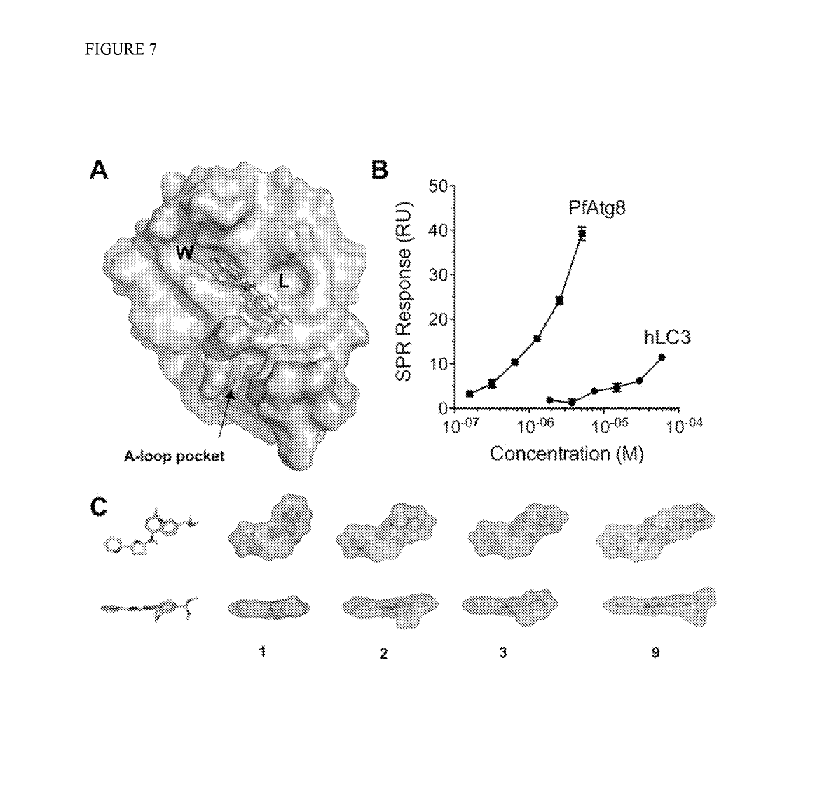

Synthesis of a novel PTA derivative with a functional handle. The present studies indicated that the PTA scaffold is a good platform for hit-optimization. A PTA-benzaldehyde derivative, 4-formyl-N-(4-pyridin-2-yl-1,3-thiazol-2-yl)benzamide (compound 7) was synthesized with a functional handle extending off the common hydrophobic ring system (Table 1, Scheme 1). Compound 7 was prepared from commercially available PTA and 4-formyl benzoic acid through a DCC-promoted amide coupling. Compound 7 can be tethered through a dialkoxyamine linker to a library of aldehydes and screened using our primary SPR competition assay against the PfAtg8-PfAtg3 interaction. In docking studies, compound 7 bound the W- and L-site of PfAtg8 in a fashion similar to compound 2, with the functional handle positioned towards the A-loop pocket (FIG. 7A). A gain in parasite selectivity for such bifunctional analogs is expected as the A-loop is missing in the human Atg8 homologues. To confirm binding to PfAtg8, compound 7 was tethered to (+)-biotinamidohexanoic acid hydrazide (BACH), through its reactive aldehyde group and tested binding with SPR. The biotinylated PTA compound 8 was immobilized onto a neutravidin coupled SPR chip. His₁₂-PfAtg8^{CM} injected at various concentrations led to a dose-dependent increase in SPR response indicating PfAtg8 directly binds compound 8 with a K_{D} of 540 nM. In contrast, the human Atg8 homologue, Microtubule-associated protein light chain 3 (hLC3) showed much lower affinity for compound 8 with a K_{D} of 18 μM, indicating specificity of the PTA scaffold for P. falciparum (FIG. 7B). Additionally, recombinant PfAtg3 did not bind immobilized compound 8, (data not shown), in agreement with our docking studies suggesting binding to the W-site of PfAtg8.

Compound 7 was converted to the hydrochloride salt and subjected to acid-catalyzed acetal formation with methanol and trimethyl orthoformate under microwave irradiation to provide the dimethyl acetal compound 9, an unreactive derivative, to confirm the inhibitory activity of the starting platform (Table 1, Scheme 1). 4-(dimethoxymethyl)-N-(4-pyridin-2-yl-1,3-thiazol-2-yl)benzamide compound 9 has similar shape and distribution of acceptor and donor pairs as the original PTA compounds (FIG. 7C) and docked onto PfAtg8 in a fashion similar to compound 7 (FIG. 7A).

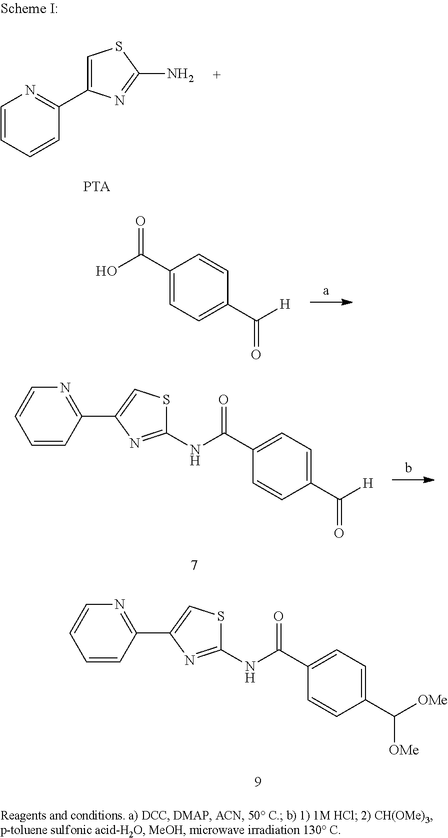 [SMILES: Nc1nc(-c2ccccn2)cs1 ; O=Cc1ccc(C(=O)O)cc1 ; O=Cc1ccc(C(=O)Nc2nc(-c3ccccn3)cs2)cc1 ; COC(OC)c1ccc(C(=O)Nc2nc(-c3ccccn3)cs2)cc1]

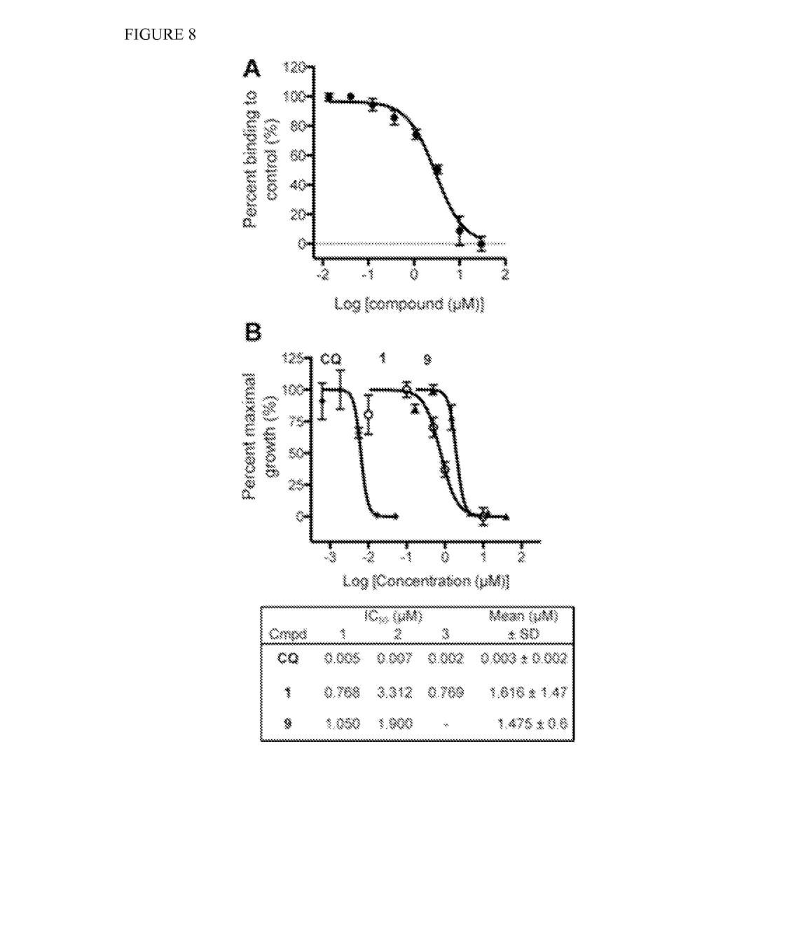

SPR studies confirmed compound 9 inhibited the PfAtg8-PfAtg3 interaction with an IC₅₀ of 2.86 μM in SPR (FIG. 8A). Growth inhibition of P. falciparum 3D7 was measured by compound 1 using the SYBR green I assay. This assay exploits the absence of nuclei in erythrocytes with a fluorescent dye that is unquenched upon binding to nucleic acids, preferentially double-stranded DNA. In two of three independent experiments, the IC₅₀ of compound 1 was 768 nM, similar to previously published results, while in a third experiment, the IC₅₀ was 3.3 μM, resulting in an average IC₅₀ of 1.61±1.47 μM. Using this assay, compound 9 had a potency similar to that of compound 1 against the blood stage of P. falciparum, with an average IC₅₀ of 1.48±0.6 μM (FIG. 8B).

Treatment of P. falciparum with high levels of compound 1 led to a drastic increase in PfAtg8 protein levels, presumably the unlipidated form as judged by its migration in SDS-PAGE. While not wishing to be limited to any particular theory, this increase could be due to an up-regulation of PfAtg8 synthesis to compensate for inhibition or a buildup of existing protein levels due to a blockade in autophagic degradation. In yeast, nitrogen starvation leads to induction of Atg8 expression while inhibition of later stages of autophagy leads to accumulation and even greater protein levels of Atg8. Further studies are necessary to determine if PfAtg8 is upregulated at the transcriptional, translational, or degradation level in response to treatment with compound 1.

The IC₅₀ in parasite cultures was much lower than against the protein-protein interaction as measured via SPR. This could be due to an accumulation of the drug inside the parasite or because even slight inhibition of PfAtg8 lipidation has drastic effects on parasite growth, similar to reaching the tipping point on a balance. An alternative explanation is the result of off-target effects; however, the observed delipidation of PfAtg8 could not be attributed to an off-target effect. Compound 1 was previously reported to have low cytotoxicity with an LD₅₀ (lethal dose) of 18.2 μM in HepG2 cells and half maximal cytotoxicity concentration (CC₅₀) and IC₅₀ of 32 μM in Huh7 cells (PubChem AID: 685525, 660872, 449705). In present invention, we did not observe cell death in HC-04 cells at 30 μM. Together, this indicates compound 1 should be a good starting scaffold for antimalarial drug design.

The inventive SPR data confirm that compound 1 directly binds to PfAtg8, while the inventive docking studies suggest the PTA scaffolds of compounds 1-3, 7, and 9 bind the W-site. The W-sites of Atg8 homologues in mammals and yeast participate in numerous protein-protein interactions through binding to the aromatic residue of an AIM, including non-autophagic proteins. Therefore, the inventive inhibitor and its derivatives can be used to identify novel apicomplexan Atg8 interactions, in various genera, as well as confirm paralogous interactions known to occur in yeast and mammalian cells.

### Examples for Compounds C5-C30

### Example 6

Virtual docking used to select 14 compounds for testing. In order to identify potential PfAtg8-PfAtg3 inhibitors, we undertook virtual library screening against the co-crystal structure of PfAtg8 with a peptide of PfAtg3 (PDB code: 4EOY). We removed the peptide and used the structure of PfAtg8 as the receptor in docking. Multiple sequence alignment and structural analysis revealed a region of Atg8, termed the A-loop, which is conserved within Apicomplexa but absent from human homologues (FIG. 9). Therefore, docking was constrained to the A-loop pocket (FIGS. 10A,B).

The ChemBridge library (July 2012) contained 369632 descriptors for small molecules, from which 10848 were considered toxic or reactive using the default filter algorithm of the program FILTER from the OpenEye suite. A property characterization of the filtered VLS library is represented in FIG. 10C. The final conformer library used for docking studies contained on average 816 conformers per small molecule, resulting in a total of 546,161,945 docking trials tested by FRED (FIG. 10D). The default Chemgauss scoring algorithm was used to rank the hits from VLS. Manual inspection of the hits resulted in the selection of 29 molecules, from which we were able to obtain 14 from commercial sources for further in vitro and in vivo parasite testing (Table 1). FIG. 10E summarizes the workflow for this selection process.

### Example 7

Surface plasmon resonance (SPR) interaction assay identifies C25 as compound to pursue. We tested the 14 VLS hits for in vitro inhibition of the plasmodial Atg8-Atg3 interaction using our established SPR competition assay (FIG. 11A). Binding is measured by SPR response of an injection of purified PfAtg8 alone or in the presence of small molecules over immobilized PfAtg3. 13 of the 14 compounds inhibited the PfAtg3-PfAtg8 interaction (FIG. 11B).

The molecules were further tested for inhibition of the homologous human interaction between hLC3 and hAtg3. None of the compounds inhibited hAtg3-hLC3 binding (FIG. 11C). We observed that some of the compounds (C11, C13, C19 and C30) either precipitated or caused protein precipitation, at the concentration used in the assay. These compounds are indicated in blue in FIG. 11 and were not further pursued. The effect of the molecules on the plasmodial and human interactions was plotted in a TSIS plot for ease of comparison (FIG. 11D).

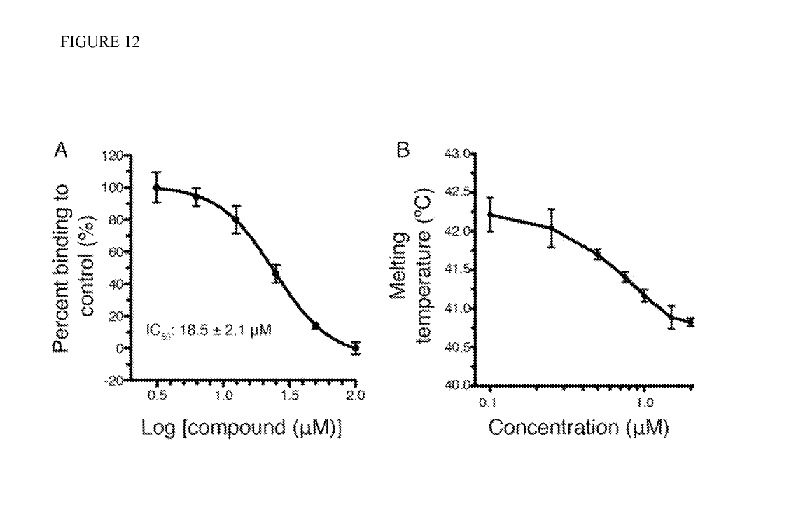

C25 and C24, the best hits, were chosen for verification of dose-dependent inhibition of the interaction. C24 led to a significant increase in binding for the human interaction, suggesting that it would not be viable as an antimalarial but may be relevant in disrupting the human interaction (by stabilization), which could have applications in cancer therapies. In terms of the plasmodial Atg8-Atg3 interaction, of the two compounds, only C25 led to dose-dependent inhibition of plasmodial Atg8-Atg3, with an SPR-IC₅₀ of 18.5±2.1 μM (FIG. 12A).

Direct binding of C25 was tested using thermal shift assays. C25 led to a dose-dependent decrease in the Tₘ of PfAtg8^{CM}, which suggests direct binding to PfAtg8, but also indicates that the compound may not be well suited for co-crystal matrix screening (FIG. 12B). Of note, C25 had the second highest ranking overall in the original docking screen. C25's docking pose and its interactions with the pocket are shown in FIG. 13.

### Example 8

C25 exhibits modest inhibition in P. falciparum cultures. We measured growth inhibition of P. falciparum 3D7 blood stage cultures by C25 using the SYBR green I assay which measures binding of a fluorescent dye to parasite DNA. In our assay, the well-established antimalarial chloroquine (CQ) had an IC₅₀ of 3±2 nM. C25 was less potent with an IC₅₀ of 19±6 μM (FIG. 14A).

In other species, drug inhibition of autophagy leads to an increase in Atg8 protein levels as Atg8 cannot be degraded and the cell up-regulates Atg8 expression. In order to confirm that C25 was affecting PfAtg8 in the parasite, we probed the lipidation status and protein level of PfAtg8 with high percent SDS-PAGE and immunoblot analysis. After a five-hour treatment with 100 μM C25, PfAtg8 protein levels increased over 10-fold compared to the loading control. Additionally, the mobility of Atg8 was retarded, indicative of the unlipidated Atg8 species (FIGS. 14B,C).

It has previously been reported that PfAtg8 partially localizes to the apicoplast during exoerythrocytic development of Plasmodium in hepatocytes. We next assessed the effect of C25 on the in vitro development of P. falciparum exoerythrocytic forms (EEFs) using a transgenic parasite strain expressing GFP throughout its life cycle. While C25 had little effect on GFP fluorescence intensity, the number of infected HC-04 cells detected by flow cytometry 96 hrs post infection was reduced by 30% in hepatocyte cultures treated with 30 μM C25 (FIG. 15A). In contrast, C25 did not affect cell viability of HC-04 cells after a 96 hour treatment (FIG. 15B).

It remains to be tested whether C25 has cross-reactivity against other apicomplexan species. However, our bioinformatics analysis suggests C25 may have activity against Eimeria, Toxoplasma/Neospora, Cryptosporidum, Theileria, and Babesia (FIG. 15E).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula I: 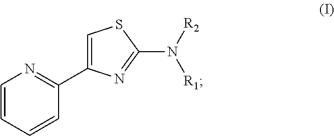
wherein R₁ is H or a C₁-C₃ alkyl, and R₂ is a substituent having the formula of formula II: 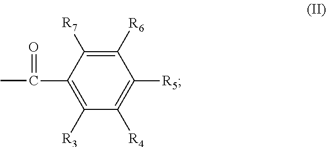
wherein R₃, R₄, R₆, and R₇ each independently represent H or C₁-C₃ alkyl, and R₅ independently represents hydrogen, C₁-C₆ alkyl, C₁-C₆ alkylamino C₁-C₆ alkyl, C₁-C₆ dialkylamino C₁-C₆ alkyl, C₁-C₆ alkylthio C₁-C₆ alkyl, C₁-C₆ alkylsulfonyl C₁-C₆ alkyl, hydroxy C₁-C₆ alkyl, C₁-C₆ alkoxy, C₁-C₆ dialkoxy, C₁-C₆ alkoxy C₁-C₆ alkyl, C3-C8 cycloalkyl, heterocyclyl, C₁-C₆ alkylamino, di C₁-C₆ alkylamino, C₁-C₆ alkylthio, C₂-C₆ alkenylthio, C₂-C₆ alkynylthio, C₂-C₆ acyloxy, thio C₂-C₆ acyl, amido, and sulphonamido, and C₁-C₆ alkyl, and C₂-C₆ alkenyl, C₂-C₆ alkynyl; wherein each alkyl moiety may be unsubstituted or substituted with one or more substituents selected from the group consisting of halo, hydroxy, carboxy, phosphoryl, phosphonyl, phosphono C₁-C₆ alkyl, carboxy C₁-C₆ alkyl, dicarboxy C₁-C₆ alkyl, C₁-C₆ dialkyl, dicarboxy halo C₁-C₆ alkyl, sulfonyl, cyano, nitro, alkoxy, alkylthio, acyl, acyloxy, thioacyl, acylthio, aryloxy, amino, alkylamino, dialkylamino, trialkylamino, arylalkylamino, guanidino, aldehydo, ureido, and aminocarbonyl; or a pharmaceutically acceptable salt, solvate, stereoisomer, or a prodrug thereof.

2. The compound of claim 1, wherein R₁, R₃, R₄, R₆, and R₇ are H.

3. The compound of claim 2, wherein R₅ is a C₁ carboxy group.

4. The compound of claim 2, wherein R₅ is a C₁ alkyl group substituted with a di methoxy group.

5. A pharmaceutical composition comprising the compound of formula I and a pharmaceutically acceptable carrier.

6. The pharmaceutical composition of claim 5, wherein R₁, R₃, R₄, R₆, and R₇ are H.

7. The pharmaceutical composition of claim 6, wherein R₅ is a C₁ carboxy group.

8. The pharmaceutical composition of claim 6, wherein R₅ is a C₁ alkyl group substituted with a dimethoxy group.

9. The pharmaceutical composition of claim 5, further comprising at least one other biologically active agent.

10. The pharmaceutical composition of claim 9, wherein the biologically active agent is an antiparasitical agent.

11. A method for inhibition of lipidation of the Atg8 protein in an apicomplexan organism comprising contacting the apicomplexan organism with an effective amount of a compound of formula I: 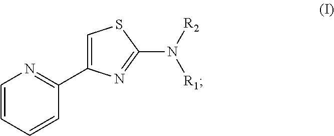
wherein R₁ is H or a C₁-C₃ alkyl, and R₂ is a substituent having the formula of formula II: 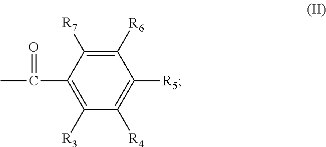
wherein R₃, R₄, R₆, and R₇ each independently represent H or C₁-C₃ alkyl, and R₅ independently represents hydrogen, C₁-C₆ alkyl, C₁-C₆ alkylamino C₁-C₆ alkyl, C₁-C₆ dialkylamino C₁-C₆ alkyl, C₁-C₆ alkylthio C₁-C₆ alkyl, C₁-C₆ alkylsulfonyl C₁-C₆ alkyl, hydroxy C₁-C₆ alkyl, C₁-C₆ alkoxy, C₁-C₆ dialkoxy, C₁-C₆ alkoxy C₁-C₆ alkyl, C3-C8 cycloalkyl, heterocyclyl, C₁-C₆ alkylamino, di C₁-C₆ alkylamino, C₁-C₆ alkylthio, C₂-C₆ alkenylthio, C₂-C₆ alkynylthio, C₂-C₆ acyloxy, thio C₂-C₆ acyl, amido, and sulphonamido, and C₁-C₆ alkyl, and C₂-C₆ alkenyl, C₂-C₆ alkynyl; wherein each alkyl moiety may be unsubstituted or substituted with one or more substituents selected from the group consisting of halo, hydroxy, carboxy, phosphoryl, phosphonyl, phosphono C₁-C₆ alkyl, carboxy C₁-C₆ alkyl, dicarboxy C₁-C₆ alkyl, C₁-C₆ dialkyl, dicarboxy halo C₁-C₆ alkyl, sulfonyl, cyano, nitro, alkoxy, alkylthio, acyl, acyloxy, thioacyl, acylthio, aryloxy, amino, alkylamino, dialkylamino, trialkylamino, arylalkylamino, guanidino, aldehydo, ureido, and aminocarbonyl; or a pharmaceutically acceptable salt, solvate, stereoisomer, or a prodrug thereof.

12. The method of claim 11, wherein the apicomplexan organism is selected from the group consisting of Plasmodium, Babesia, Cryptosporidium, Clyclospora, Isospora, Eimeria, Theileria and Toxoplasma.

13. The method of claim 12, wherein the organism is Plasmodium falciparum, or Plasmodium vivax.

14. A method of treatment of a apicomplexan infection in a subject in need thereof comprising administering to the subject an effective amount of one or more of the pharmaceutical compositions of claim 5.

15. A method of treatment of a apicomplexan infection in a subject in need thereof comprising administering to the subject an effective amount of one or more of the pharmaceutical compositions of claim 5, and at least one additional biologically active agent.

16. A pharmaceutical composition comprising one or more of the following compounds selected from the group consisting of: 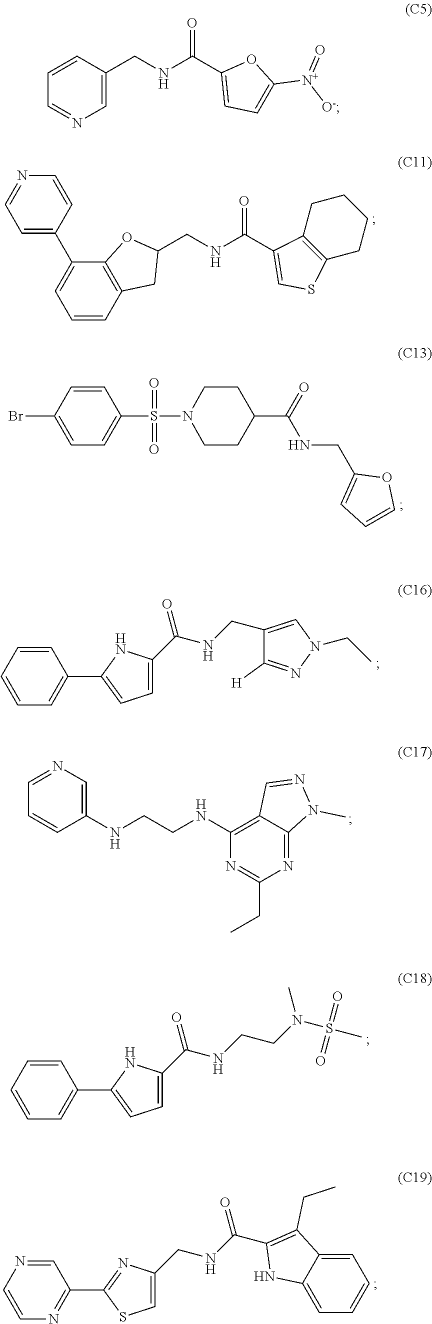 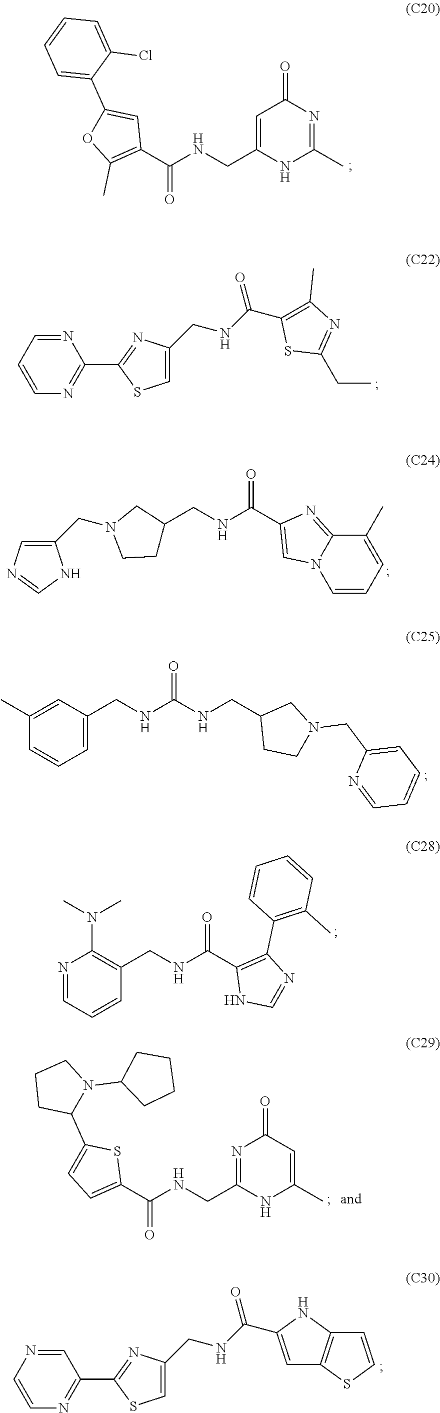
or a pharmaceutically acceptable salt, solvate, stereoisomer, or a prodrug thereof, and a pharmaceutically acceptable carrier.

17. A method for the inhibition of lipidation of the Atg8 protein in an apicomplexan organism comprising contacting the apicomplexan organism with an effective amount of one or more of the pharmaceutical compositions of claim 16.

18. The use method of claim 17, wherein the apicomplexan organism is selected from the group consisting of Plasmodium, Babesia, Cryptosporidium, Clyclospora, Isospora, Eimeria, Theileria and Toxoplasma.

19. A method for treatment of an apicomplexan infection in a subject in need thereof comprising administering to the subject an effective amount of one or more of the pharmaceutical compositions of claim 16.

20. The method of claim 19 further comprising the administration to the subject of at least one additional biologically active agent, in an effective amount.

21. The method of claim 19, wherein the apicomplexan organism is selected from the group consisting of Plasmodium, Babesia, Cryptosporidium, Cyclospora, Isospora, Eimeria, Theileria and Toxoplasma.

22. The method of claim 20, wherein the apicomplexan organism is selected from the group consisting of Plasmodium, Babesia, Cryptosporidium, Cyclospora, Isospora, Eimeria, Theileria and Toxoplasma.
